(19) United States
(12) Patent Application Publication
(10) Pub. No.: US 2009/0259112 A1
(43) Pub. Date: Oct. 15, 2009

(54) Sensors

(75) Inventors: Roderick A. Hyde, Redmond, WA (US); Muriel Y. Ishikawa, Livermore, CA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/082,404

(22) Filed: Apr. 9, 2008

Related U.S. Application Data
(63) Continuation-in-part of application No. 12/082,402, filed on Apr. 9, 2008.
Application No. 12/082,402 is a continuation-in-part of application No. 12/082,403, filed on Apr. 9, 2008.

(51) Int. Cl. A61B 5/00

(52) U.S. Cl. 600/300; 600/301

(57) ABSTRACT

The present disclosure relates to sensors and systems associated with the sensors.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. UNKNOWN, entitled METHODS AND SYSTEMS ASSOCIATED WITH DELIVERY OF ONE OR MORE AGENTS TO AN INDIVIDUAL, naming Roderick A. Hyde, Muriel Y. Ishikawa, and Lowell L. Wood, Jr. as inventors, filed 9 Apr. 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. UNKNOWN, entitled AGENT DELIVERY DEVICE, naming Roderick A. Hyde, Muriel Y. Ishikawa, and Lowell L. Wood, Jr. as inventors, filed 9 Apr. 2008, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present disclosure relates to sensors and systems associated with the sensors.

### SUMMARY

In one aspect, a sensor includes but is not limited to one or more sensor housings that include one or more selectively accessible sections, one or more detectors operably associated with the one or more selectively accessible sections, one or more barriers operably associated with the one or more selectively accessible sections, one or more sensor control units, and one or more sensor transmitters. The sensor may optionally include one or more sensor receivers. In addition to the foregoing, other aspects are described in the claims, drawings, and text forming a part of the present disclosure.

In one aspect, a system includes but is not limited to circuitry for operating one or more sensor housings that include one or more selectively accessible sections, circuitry for operating one or more detectors operably associated with the one or more selectively accessible sections, circuitry for operating one or more barriers operably associated with the one or more selectively accessible sections, circuitry for operating one or more sensor control units, and circuitry for operating one or more sensor transmitters. The system may optionally include circuitry for operating one or more sensor receivers. In addition to the foregoing, other system aspects are described in the claims, drawings, and text forming a part of the present disclosure.

In one aspect, a system includes but is not limited to means for operating one or more sensor housings that include one or more selectively accessible sections, means for operating one or more detectors operably associated with the one or more selectively accessible sections, means for operating one or more barriers operably associated with the one or more selectively accessible sections, means for operating one or more sensor control units, and means for operating one or more sensor transmitters. The system may optionally include means for operating one or more sensor receivers. In addition to the foregoing, other system aspects are described in the claims, drawings, and text forming a part of the present disclosure.

In one aspect, a system includes but is not limited to a signal-bearing medium bearing one or more instructions for operating one or more sensor housings that include one or more selectively accessible sections, one or more instructions for operating one or more detectors operably associated with the one or more selectively accessible sections, one or more instructions for operating one or more sensor control units, and one or more instructions for operating one or more sensor transmitters. The system may optionally include one or more instructions for operating one or more sensor receivers. In addition to the foregoing, other system aspects are described in the claims, drawings, and text forming a part of the present disclosure.

In one or more various aspects, means include but are not limited to circuitry and/or programming for effecting the herein referenced functional aspects; the circuitry and/or programming can be virtually any combination of hardware, software, and/or firmware configured to effect the herein referenced functional aspects depending upon the design choices of the system designer. In addition to the foregoing, other system aspects means are described in the claims, drawings, and/or text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein referenced method aspects depending upon the design choices of the system designer. In addition to the foregoing, other system aspects are described in the claims, drawings, and/or text forming a part of the present application.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 illustrates an example system 100 in which embodiments may be implemented.

FIG. 2 illustrates an embodiment of a sensor.

FIG. 3 illustrates alternate embodiments of the sensor of FIG. 2.

FIG. 4 illustrates alternate embodiments of the sensor of FIG. 2.

FIG. 5 illustrates alternate embodiments of the sensor of FIG. 2.

FIG. 6 illustrates alternate embodiments of the sensor of FIG. 2.

FIG. 7 illustrates alternate embodiments of the sensor of FIG. 2.

FIG. 8 illustrates alternate embodiments of the sensor of FIG. 2.

FIG. 9 illustrates alternate embodiments of the sensor of FIG. 2.

FIG. 10 illustrates alternate embodiments of the sensor of FIG. 2.

FIG. 11 illustrates alternate embodiments of the sensor of FIG. 2.

FIG. 12 illustrates alternate embodiments of the sensor of FIG. 2.

FIG. 13 illustrates alternate embodiments of the sensor of FIG. 2.

FIG. 14 illustrates alternate embodiments of the sensor of FIG. 2.

FIG. 15 illustrates alternate embodiments of the sensor of FIG. 2.

FIG. 16 illustrates an embodiment of a sensor.

FIG. 17 illustrates alternate embodiments of the sensor of FIG. 16.

FIG. 18 illustrates alternate embodiments of the sensor of FIG. 16.

FIG. 19 illustrates alternate embodiments of the sensor of FIG. 16.

FIG. 20 illustrates a partial view of a system 2000 that includes a computer program for executing a computer process on a computing device.

FIG. 21 illustrates a partial view of a system 2100 that includes a computer program for executing a computer process on a computing device.

FIG. 22A illustrates an embodiment of an agent delivery device.

FIG. 22B illustrates an embodiment of an agent delivery device.

FIG. 23A illustrates an embodiment of an agent delivery device.

FIG. 23B illustrates an embodiment of an agent delivery device.

FIG. 24A illustrates an embodiment of an agent delivery device.

FIG. 24B illustrates an embodiment of an agent delivery device.

FIG. 25A illustrates an embodiment of an agent delivery device.

FIG. 25B illustrates an embodiment of an agent delivery device.

FIG. 26 illustrates an embodiment of an agent delivery device.

FIG. 27 illustrates an embodiment of an agent delivery device.

FIG. 28A illustrates an embodiment of an agent delivery device.

FIG. 28B illustrates an embodiment of an agent delivery device.

FIG. 29A illustrates an embodiment of an agent delivery device.

FIG. 29B illustrates an embodiment of an agent delivery device.

FIG. 30A illustrates an embodiment of an agent delivery device.

FIG. 30B illustrates an embodiment of an agent delivery device.

FIG. 31A illustrates an embodiment of an agent delivery device.

FIG. 31B illustrates an embodiment of an agent delivery device.

FIG. 32A illustrates an embodiment of an agent delivery device.

FIG. 32B illustrates an embodiment of an agent delivery device.

FIG. 33A illustrates an embodiment of a sensor.

FIG. 33B illustrates an embodiment of a sensor.

FIG. 33C illustrates an embodiment of a sensor.

FIG. 34A illustrates an embodiment of a sensor.

FIG. 34B illustrates an embodiment of a sensor.

FIG. 34C illustrates an embodiment of a sensor.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

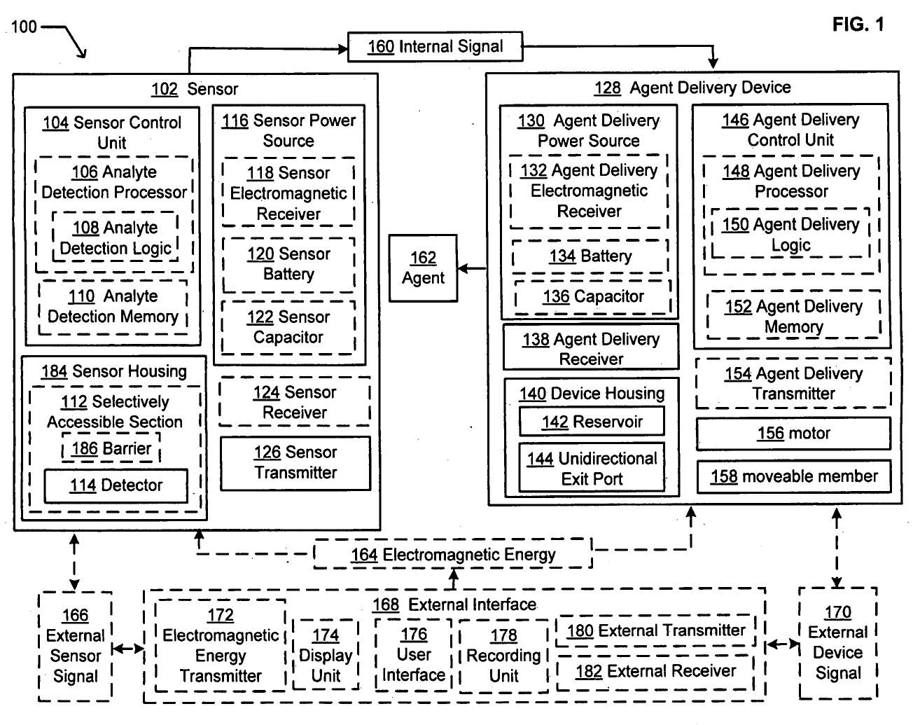

FIG. 1 illustrates an example system 100 in which embodiments may be implemented. In some embodiments, system 100 may include one or more sensors 102. In some embodiments, system 100 may include one or more agent delivery devices 128. In some embodiments, system 100 may include one or more external interfaces 168. In some embodiments, one or more sensors 102 may be configured to transmit one or more internal signals 160. In some embodiments, one or more agent delivery devices 128 may be configured to receive one or more internal signals 160. In some embodiments, one or more agent delivery devices 128 may be configured to administer one or more agents 162. In some embodiments, one or more external interfaces 168 may be configured to transmit electromagnetic energy 164. In some embodiments, one or more external interfaces 168 may be configured to transmit one or more external sensor signals 166. In some embodiments, one or more external interfaces 168 may be configured to transmit one or more external device signals 170.

In some embodiments, one or more agent delivery devices 128 may include one or more agent delivery receivers 138 that may be configured to receive one or more internal signals 160. In some embodiments, one or more agent delivery devices 128 may include one or more agent delivery receivers 138 that may be configured to receive one or more external device signals 170. In some embodiments, one or more agent delivery devices 128 may include one or more agent delivery transmitters 154 that may be configured to transmit one or more external device signals 170. In some embodiments, one or more agent delivery devices 128 may include one or more agent delivery power sources 130. In some embodiments, one or more agent delivery devices 128 may include one or more agent delivery electromagnetic receivers 132 that may be configured to receive electromagnetic energy 164. In some embodiments, one or more agent delivery devices 128 may include one or more batteries 134. In some embodiments, one or more agent delivery devices 128 may include one or more capacitors 136. In some embodiments, one or more agent delivery devices 128 may include one or more device housings 140. In some embodiments, one or more agent delivery devices 128 may include one or more reservoirs 142. In some embodiments, one or more agent delivery devices 128 may include one or more unidirectional exit ports 144. In some embodiments, one or more agent delivery devices 128 may include one or more agent delivery control units 146. In some embodiments, one or more agent delivery devices 128 may include one or more agent delivery processors 148. In some embodiments, one or more agent delivery devices 128 may include agent delivery logic 150. In some embodiments, one or more agent delivery devices 128 may include agent delivery memory 152. In some embodiments, one or more agent delivery devices 128 may include one or more motors 156. In some embodiments, one or more agent delivery devices 128 may include one or more moveable members 158.

In some embodiments, one or more sensors 102 may include one or more sensor transmitters 126 that may be configured to transmit one or more internal signals 160. In some embodiments, one or more sensors 102 may include one or more sensor transmitters 126 that may be configured to transmit one or more external sensor signals 166. In some embodiments, one or more sensors 102 may include one or more sensor receivers 124 that may be configured to receive one or more external sensor signals 166. In some embodiments, one or more sensors 102 may include one or more sensor power sources 116. In some embodiments, one or more sensors 102 may include one or more sensor electromagnetic receivers 118 that may be configured to receive electromagnetic energy 164. In some embodiments, one or more sensors 102 may include one or more sensor batteries 120. In some embodiments, one or more sensors 102 may include one or more sensor capacitors 122. In some embodiments, one or more sensors 102 may include one or more sensor control units 104. In some embodiments, one or more sensors 102 may include one or more analyte detection processors 106. In some embodiments, one or more sensors 102 may include analyte detection logic 108. In some embodiments, one or more sensors 102 may include analyte detection memory 110. In some embodiments, one or more sensors 102 may include one or more selectively accessible sections 112. In some embodiments, one or more sensors 102 may include one or more detectors 114.

In some embodiments, system 100 may include one or more external interfaces 168 that include one or more external receivers 182 that are configured to receive one or more external sensor signals 166. In some embodiments, system 100 may include one or more external interfaces 168 that include one or more external receivers 182 that are configured to receive one or more external device signals 170. In some embodiments, system 100 may include one or more external interfaces 168 that include one or more external transmitters 180 that are configured to transmit one or more external sensor signals 166. In some embodiments, system 100 may include one or more external interfaces 168 that include one or more external transmitters 180 that are configured to transmit one or more external device signals 170. In some embodiments, system 100 may include one or more external interfaces 168 that include one or more electromagnetic energy transmitters 172 that are configured to transmit electromagnetic energy 164. In some embodiments, system 100 may include one or more external interfaces 168 that include one or more user interfaces 176. In some embodiments, system 100 may include one or more external interfaces 168 that include one or more display units 174. In some embodiments, system 100 may include one or more external interfaces 168 that include one or more recording units 178.

### Sensor

System 100 may include one or more sensors 102. In some embodiments, one or more sensors 102 may be configured for implantation within an individual (e.g., U.S. Pat. Nos. 7,110,803 and 7,044,911). Sensors 102 may be configured for implantation at numerous positions within an individual. For example, in some embodiments, one or more sensors 102 may be configured for implantation into the vasculature of an individual (e.g., U.S. Pat. Nos. 7,181,261; 7,025,734; and 7,236,821).

A sensor 102 may be operably associated with one or more sensor control units 104. In some embodiments, the one or more sensor control units 104 may serve to regulate the activity of one or more sensors 102. For example, in some embodiments, one or more sensor control units 104 may regulate one or more times when one or more sensors 102 detect one or more analytes. In some embodiments, one or more sensor control units 104 may regulate one or more time periods when one or more sensors 102 detect one or more analytes. In some embodiments, one or more sensor control units 104 may regulate what analytes are detected by one or more sensors 102. In some embodiments, one or more sensor control units 104 may regulate unmasking of one or more selectively accessible sections 112 of one or more sensors 102. For example, in some embodiments, one or more sensor control units 104 may regulate unmasking of selectively accessible sections 112 of one or more sensors 102 to expose one or more detectors 114 at one or more times. Accordingly, in some embodiments, one or more sensor control units 104 may regulate which detectors 114 are available for detection of one or more analytes and when the one or more detectors 114 are made available. In some embodiments, one or more sensor control units 104 may be operably coupled to one or more analyte detection processors 106. In some embodiments, one or more sensors 102 may include an analyte detection processor 106 that is configured to process information received from one or more detectors 114. For example, in some embodiments, one or more analyte detection processors 106 may be configured to calculate the concentration of one or more detected analytes. In some embodiments, one or more analyte detection processors 106 may be configured to determine changes in the concentration of one or more detected analytes relative to time. In some embodiments, one or more analyte detection processors 106 may be configured to determine changes in the concentration of one or more detected analytes relative to one or more amounts of agent 162 that are administered to an individual. In some embodiments, one or more analyte detection processors 106 may be configured to prepare one or more instructions for one or more agent delivery devices 128. For example, in some embodiments, one or more analyte detection processors 106 may instruct one or more agent delivery devices 128 to administer one or more amounts of one or more agents 162. In some embodiments, one or more analyte detection processors 106 may instruct one or more agent delivery devices 128 to administer one or more agents 162 at one or more times. In some embodiments, one or more analyte detection processors 106 may instruct one or more agent delivery devices 128 to administer one or more amounts of one or more agents 162 at one or more times. In some embodiments, one or more analyte detection processors 106 may include analyte detection logic 108. For example, in some embodiments, one or more analyte detection processors 106 may include analyte detection logic 108 that is programmed to compensate for background occurring during detection of one or more analytes. In some embodiments, analyte detection logic 108 may be configured to process information obtained during detection of one or more analytes to account for the personal characteristics of the individual into which the sensor 102 is implanted. For example, in some embodiments, analyte detection logic 108 may be configured to determine the amount of one or more agents 162 to be administered to an individual to maintain the concentration of the one or more agents 162 at one or more setpoints within the individual. In some embodiments, analyte detection logic 108 may be configured to determine the amount of one or more agents 162 to be administered to an individual to maintain the concentration of the one or more agents 162 within one or more concentration ranges within the individual. In some embodiments, a sensor control unit 104 may include analyte detection memory 110. For example, in some embodiments, one or more sensors 102 may save information associated with the identity of one or more detected analytes, the identity of one or more undetected analytes, the concentration of one or more analytes, changes in the concentration of one or more analytes, or substantially any combination thereof. Numerous types of memory may be used for analyte detection memory 110. Examples of memory include, but are not limited to, flash memory, random access memory, read-only memory, and the like.

In some embodiments, a sensor 102 may include one or more sensor housings 184. In some embodiments, one or more sensor housings 184 may be operably coupled with one or more detectors 114. In some embodiments, one or more sensor housings 184 may include one or more selectively accessible sections 112. In some embodiments, one or more sensor housings 184 may include one or more selectively accessible sections 112 that enclose one or more detectors 114. In some embodiments, one or more selectively accessible sections 112 may include one or more structures that modulate access to the one or more selectively accessible sections 112 of the sensor housing 184. For example, in some embodiments, one or more selectively accessible sections 112 may be covered with a gold sacrificial layer that may be removed through electrochemical dissolution with a constant DC current (e.g., 35 mA/cm²) (Pan et al., Proceedings of the 26^{th} Annual International Conference of the IEEE EMBS, San Francisco, Calif., USA, Sep. 1-5, 2004). In some embodiments, one or more selectively accessible sections 112 may be covered with a shape-memory polymer that may be activated to unsequester the one or more selectively accessible sections 112 (e.g., U.S. Pat. No. 6,454,759).

In some embodiments, the one or more selectively accessible sections 112 may sequester one or more detectors 114 that may be selectively unsequestered. Numerous types of detectors 114 may be associated with one or more sensors 102. In some embodiments, numerous different types of detectors 114 may be associated with one or more sensors 102. Examples of such detectors 114 include, but are not limited to, electrodes, surface plasmon resonance detectors 114, microelectromechanical systems detectors 114, microcantilever detectors 114, nitric oxide detectors 114, osmotic detectors 114, relativity-based detectors 114, chemical detectors 114, pressure detectors 114, electrochemical detectors 114, piezoelectric detectors 114, pH detectors 114, hydrogel detectors 114, enzymatic detectors 114, ball integrated circuit detectors 114, affinity viscosimetric detectors 114, blood pressure detectors 114; metal detectors 114, glucose detectors 114, and the like (e.g., U.S. Pat. Nos. 7,162,289; 6,280,604; 5,603,820; 5,582,170; 6,287,452; 7,291,503; 6,764,446; 7,168,294; 6,823,717; 7,205,701; 6,268,161; 4,703,756; 6,965,791; 6,546,268; 6,210,326; 6,514,689; 6,234,973; 6,442,413; Tu et al., Electroanalysis, 11:70-74 (1999), Malinski et al., Molecular Mechanisms of Metal Toxicity and Carcinogenicity, Environmental Health Perspectives 102, Supplement 3, September 1994). In some embodiments, one or more detectors 114 may be configured to detect one or more agents 162. Examples of such agents include, but are not limited to, pharmaceutical agents 162, hormones, cytokines, and the like. In some embodiments, one or more detectors 114 may be configured to detect one or more sugars (e.g., glucose). In some embodiments, one or more detectors 114 may be configured to detect one or more pathogen indicators (e.g., viruses, molds, bacteria, fungi, parasites, worms, eggs, pathogen associated products, pathogen associated components, etc.). In some embodiments, one or more detectors 114 may be configured to detect one or more cancer markers. Examples of such cancer markers include, but are not limited to, cancer antigen 125 (ovarian cancer), CA 15.3 (breast and ovarian cancer), CA 27.29 (breast cancer), carcinoembryonic antigen (colorectal cancer, gastric cancer, pancreatic cancer, lung cancer, breast cancer), carbohydrate antigen 19-9 (pancreatic cancer), neuron-specific enolase (neuroblastoma, small cell lung cancer, medullary thyroid cancer, carcinoid tumors, pancreatic endocrine tumors, and melanoma), carcinoembryonic antigen (intestinal cancer), lactate dehydrogenase (testicular cancer, Ewing's sarcoma, non-Hodgkin's lymphoma, leukemia), HER2 (breast cancer), prostate-specific antigen (prostate cancer), acid phosphatase (prostate cancer), alpha-fetoprotein (hepatocellular carcinoma), and the like.

In some embodiments, one or more sensor housings 184 may include circuitry that is operably coupled to one or detectors 114. In some embodiments, one or more sensor housings 184 may include circuitry that is configured to facilitate elimination of one or more sacrificial layers. In some embodiments, one or more sensor housings 184 may include circuitry that is configured to facilitate reconfiguration of one or more shape memory materials. In some embodiments, one or more sensor housings 184 may include circuitry that is configured to be operably coupled to one or more detectors 114. In some embodiments, one or more sensor housings 184 may include circuitry that is configured to be operably coupled to one or more sensor control units 104. In some embodiments, one or more sensor housings 184 may include circuitry that is configured to be operably coupled to one or more sensor power sources 116. In some embodiments, one or more sensor housings 184 may include circuitry that is configured to be operably coupled to one or more sensor receivers 124. In some embodiments, one or more sensor housings 184 may include circuitry that is configured to be operably coupled to one or more sensor transmitters 126.

In some embodiments, a sensor 102 may include one or more sensor power sources 116. In some embodiments, a sensor 102 may be operably coupled to one or more sensor batteries 120. In some embodiments, a sensor battery 120 may include a thin-film fuel cell for providing electrical power. In some embodiments, the fuel cell may be of a solid oxide type (SOFC), a solid polymer type (SPFC), a proton exchange membrane type (PEMFC), and/or substantially any combination thereof. Methods to fabricate such thin-film fuel cells are known and have been described (e.g., U.S. Pat. No. 7,189,471). In some embodiments, one or more sensor batteries 120 may include one or more storage films that are configured for energy storage and energy conversion. Methods to fabricate such storage films are known and have been described (e.g., U.S. Pat. No. 7,238,628). In some embodiments, a sensor battery 120 may be a biobased battery (e.g., U.S. Pat. No. 6,994,934). In some embodiments, one or more sensor batteries 120 may be thin-film batteries. Methods to fabricate thin-film batteries are known and have been described (e.g., U.S. Pat. Nos. 7,194,801; 7,144,655; 6,818,356). In some embodiments, one or more sensor electromagnetic receivers 118 may be used to electromagnetically couple power to energize one or more sensors 102 from an external power source. Methods to construct electromagnetic receivers have been described (e.g., U.S. Pat. No. 5,571,152). Briefly, in some embodiments, one or more electromagnetic receivers may be associated with one or more rectifier chips. The one or more sensor electromagnetic receivers 118 may include one or more cores about which are wrapped an electrical conductor. In some embodiments, cores may comprise a material, such as a ferrite material, due to its relatively high magnetic permeability and low magnetic hysteresis. However, other materials can be used for this purpose. In some embodiments, a sensor 102 may be operably coupled to one or more sensor capacitors 122. In some embodiments, one or more sensor electromagnetic receivers 118 may be operably coupled to one or more batteries. In some embodiments, one or more sensor electromagnetic receivers 118 may be operably coupled to one or more sensor capacitors 122. Accordingly, in some embodiments, one or more sensors 102 may be configured such that they are operably coupled to a rechargeable power source.

The system 100 may include one or more sensor transmitters 126. Numerous types of sensor transmitters 126 may be used in association with system 100. Examples of such sensor transmitters 126 include, but are not limited to, transmitters that transmit one or more acoustic signals, optical signals, radio signals, wireless signals, hardwired signals, infrared signals, ultrasonic signals, and the like (e.g., U.S. Pat. Nos: RE39,785; 7,260,768; 7,260,764; 7,260,402; 7,257,327; 7,215,887; 7,218,900). In some embodiments, one or more sensor transmitters 126 may transmit one or more signals that are encrypted. Numerous types of transmitters are known and have been described (e.g., U.S. Pat. Nos. and Published U.S. Patent Applications: 7,236,595; 7,260,155; 7,227,956; US2006/0280307).

The system 100 may include one or more sensor receivers 124. Numerous types of sensor receivers 124 may be used in association with system 100. Examples of such sensor receivers 124 include, but are not limited to, receivers that receive one or more acoustic signals, optical signals, radio signals, wireless signals, hardwired signals, infrared signals, ultrasonic signals, and the like. Such receivers are known and have been described (e.g., U.S. Pat. Nos. RE39,785; 7,218,900; 7,254,160; 7,245,894; 7,206,605).

### Agent Delivery Device

The system 100 may include one or more agent delivery devices 128. In some embodiments, an agent delivery device 128 may be configured for implantation within an individual. In some embodiments, an agent delivery device 128 may include one or more agent delivery power sources 130. In some embodiments, an agent delivery device 128 may be operably coupled to one or more batteries 134. In some embodiments, a battery 134 may include a thin-film fuel cell for providing electrical power. In some embodiments, the fuel cell may be of a solid oxide type (SOFC), a solid polymer type (SPFC), a proton exchange membrane type (PEMFC), and/or substantially any combination thereof. Methods to fabricate such thin-film fuel cells are known and have been described (e.g., U.S. Pat. No. 7,189,471). In some embodiments, one or more batteries 134 may include one or more storage films that are configured for energy storage and energy conversion. Methods to fabricate such storage films are known and have been described (e.g., U.S. Pat. No. 7,238,628). In some embodiments, a battery 134 may be a biobased battery 134 (e.g., U.S. Pat. No. 6,994,934). In some embodiments, one or more batteries 134 may be thin-film batteries 134. Methods to fabricate thin-film batteries 134 are known and have been described (e.g., U.S. Pat. Nos. 7,194,801; 7,144,655; 6,818,356). In some embodiments, one or more agent delivery electromagnetic receivers 132 may be used to electromagnetically couple power to energize one or more agent delivery devices 128 from an external power source. Methods to construct electromagnetic receivers have been described (e.g., U.S. Pat. No. 5,571,152). Briefly, in some embodiments, one or more electromagnetic receivers may be associated with one or more rectifier chips. The one or more agent delivery electromagnetic receivers 132 may include one or more cores about which are wrapped an electrical conductor. In some embodiments, cores may comprise a material, such as a ferrite material, due to its relatively high magnetic permeability and low magnetic hysteresis. However, other materials can be used for this purpose. In some embodiments, an agent delivery device 128 may be operably coupled to one or more capacitors 136. In some embodiments, one or more agent delivery electromagnetic receivers 132 may be operably coupled to one or more batteries 134. In some embodiments, one or more agent delivery electromagnetic receivers 132 may be operably coupled to one or more capacitors 136. Accordingly, in some embodiments, one or more agent delivery devices 128 may be configured such that they are operably coupled to a rechargeable power source.

An agent delivery device 128 may be operably associated with one or more agent delivery control units 146. In some embodiments, the one or more agent delivery control units 146 may serve to regulate the activity of one or more agent delivery devices 128. For example, in some embodiments, one or more agent delivery control units 146 may regulate one or more times when one or more agent delivery devices 128 administer one or more agents 162. In some embodiments, one or more agent delivery control units 146 may regulate one or more time periods when one or more agent delivery devices 128 administer one or more agents 162. In some embodiments, one or more agent delivery control units 146 may regulate what agents 162 are administered by one or more agent delivery devices 128. In some embodiments, one or more agent delivery control units 146 may regulate the operation of one or more motors 156 associated with one or more agent delivery devices 128. For example, in some embodiments, one or more agent delivery control units 146 may regulate the duration of operation of one or more motors 156. In some embodiments, one or more agent delivery control units 146 may regulate the time when one or more motors 156 are operated. In some embodiments, one or more agent delivery control units 146 may regulate the frequency with which one or more motors 156 are operated. In some embodiments, one or more agent delivery control units 146 may be operably coupled to one or more agent delivery processors 148. In some embodiments, one or more agent delivery devices 128 may include an agent delivery processor 148 that is configured to process information received from one or more sensors 102. For example, in some embodiments, one or more agent delivery processors 148 may be configured to calculate the concentration of one or more detected analytes. In some embodiments, one or more agent delivery processors 148 may be configured to determine changes in the concentration of one or more detected analytes relative to time. In some embodiments, one or more agent delivery processors 148 may be configured to determine changes in the concentration of one or more detected analytes relative to one or more amounts of agent 162 that are administered to an individual. In some embodiments, one or more agent delivery processors 148 may be configured to regulate one or more motors 156 that are operably coupled to the agent delivery device 128. For example, in some embodiments, one or more agent delivery processors 148 may facilitate operation of one or more motors 156 to administer one or more amounts of one or more agents 162. In some embodiments, one or more agent delivery processors 148 may facilitate operation of one or more motors 156 to administer one or more agents 162 at one or more times. In some embodiments, one or more agent delivery processors 148 may facilitate operation of one or more motors 156 to administer one or more amounts of one or more agents 162 at one or more times. In some embodiments, one or more agent delivery processors 148 may include agent delivery logic 150. For example, in some embodiments, one or more agent delivery processors 148 may include agent delivery logic 150 that is programmed to facilitate administration of one or more agents 162 to an individual. In some embodiments, one or more agent delivery processors 148 may include agent delivery logic 150 that is programmed to facilitate administration of one or more agents 162 to an individual such that the concentration of the one or more agents 162 is substantially maintained at a setpoint. In some embodiments, one or more agent delivery processors 148 may include agent delivery logic 150 that is programmed to facilitate administration of one or more agents 162 to an individual such that the concentration of the one or more agents 162 is substantially maintained within a range of concentrations. In some embodiments, one or more agent delivery processors 148 may include agent delivery logic 150 that is programmed to facilitate administration of one or more agents 162 to an individual with regard to characteristics of the individual. For example, in some embodiments, agent delivery logic 150 may account for the size of an individual to facilitate administration of one or more agents 162 to an individual. In some embodiments, an agent delivery control unit 146 may include agent delivery memory 152. For example, in some embodiments, one or more agent delivery devices 128 may save information associated with the identity of one or more administered agents 162, the concentration of one or more administered agents 162, changes in the concentration of one or more agents 162, or substantially any combination thereof. Numerous types of memory may be used for agent delivery memory 152. Examples of memory include, but are not limited to, flash memory, random access memory, read-only memory, and the like.

An agent delivery device 128 may include one or more agent delivery transmitters 154. Numerous types of agent delivery transmitters 154 may be used in association with system 100. Examples of such agent delivery transmitters 154 include, but are not limited to, transmitters that transmit one or more acoustic signals, optical signals, radio signals, wireless signals, hardwired signals, infrared signals, ultrasonic signals, and the like (e.g., U.S. Pat. Nos. RE39,785; 7,260,768; 7,260,764; 7,260,402; 7,257,327; 7,215,887; 7,218,900; herein incorporated by reference). In some embodiments, one or more agent delivery transmitters 154 may transmit one or more signals that are encrypted. Numerous types of transmitters are known and have been described (e.g., U.S. Pat. Nos. and Published U.S. Patent Applications: 7,236,595; 7,260,155; 7,227,956; US2006/0280307; herein incorporated by reference).

An agent delivery device 128 may include one or more agent delivery receivers 138. Numerous types of agent delivery receivers 138 may be used in association with system 100. Examples of such agent delivery receivers 138 include, but are not limited to, receivers that receive one or more acoustic signals, optical signals, radio signals, wireless signals, hardwired signals, infrared signals, ultrasonic signals, and the like. Such receivers are known and have been described (e.g., U.S. Pat. Nos. RE39,785; 7,218,900; 7,254,160; 7,245,894; 7,206,605; herein incorporated by reference).

An agent delivery device 128 may include one or more device housings 140. In some embodiments, one or more device housings 140 may include one or more reservoirs 142. In some embodiments, one or more device housings 140 may be operably coupled with one or more unidirectional exit ports 144. In some embodiments, one or more device housings 140 may be operably coupled with one or more motors 156. In some embodiments, one or more device housings 140 may be operably coupled with one or more moveable members 158. For example, in some embodiments, a device housing 140 may be configured as a tube with a unidirectional exit port 144 operably coupled to a distal end of the tube. In some embodiments, such a device housing 140 may be configured to accept a moveable member 158 that is configured to slide within the interior of the device housing tube from a proximal end of the tube to the distal end of the tube. In some embodiments, the moveable member 158 may be operably associated with one or more motors 156 that are configured to translocate the moveable member 158. In some embodiments, the space within the tube between the moveable member 158 and the unidirectional exit port 144 may be configured as a reservoir 142 that may include one or more agents 162. Accordingly, movement of the moveable member 158 from the proximal end to the distal end of the tube will cause the one or more agents 162 to be expelled from the unidirectional exit port 144. Numerous types of motors 156 may be associated with one or more agent delivery devices 128. Examples of such motors 156 include, but are not limited to, stepper motors 156, osmotic motors 156, piezoelectric motors 156, ultrasonic motors 156, acoustic motors 156, and the like. In some embodiments, one or more moveable members 158 may be operably associated with one or more ratcheted members such that the one or more moveable members 158 may be engaged by the one or more ratcheted members in conjunction with movement facilitated by one or more motors 156.

### Signal

Numerous types of signals may be used in association with system 100. In some embodiments, a signal may be an internal signal 160. In some embodiments, a signal may be an external sensor signal 166. In some embodiments, a signal may be an external device signal 170. Examples of such signals include, but are not limited to, analog signals, digital signals, acoustic signals, optical signals, radio signals, wireless signals, hardwired signals, infrared signals, ultrasonic signals, and the like. In some embodiments, one or more signals may not be encrypted. In some embodiments, one or more signals may be encrypted. In some embodiments, one or more signals may be sent through use of a secure mode of transmission. In some embodiments, one or more signals may be coded for receipt by a specific individual. In some embodiments, such code may include anonymous code that is specific for an individual. Accordingly, information included within one or more signals may be protected against being accessed by others who are not the intended recipient.

In some embodiments, one or more signals may include information associated with the operation of one or more agent delivery devices 128. In some embodiments, one or more signals may include information associated with the operation of one or more motors 156 associated with an agent delivery device 128. For example, in some embodiments, one or more signals may include information associated with the operation of one or more stepper motors 156 associated with an agent delivery device 128. Examples of such information include, but are not limited to, the number of cycles that a motor 156 is to operate, the number of steps that a motor 156 is to operate, the duration of time for which a motor 156 is to operate, the rate at which a motor 156 is to operate, one or more times when a motor 156 is to operate, and the like. Such information may be associated with numerous types of motors 156. In some embodiments, one or more signals may include information that is associated with the operation of one or more ports that are associated with one or more agent delivery devices 128. In some embodiments, one or more signals may include instructions for an agent delivery device 128 to open one or more ports. In some embodiments, one or more signals may include instructions for an agent delivery device 128 to close one or more ports. Examples of such ports include, but are not limited to, electromagnetic ports, shape memory ports, and the like (e.g., Low et al., Sensors and Actuators B: Chemical, 76:149-160 (2000), Pan et al., Proceedings of the 26^{th} Annual International Conference of the IEEE EMBS, San Francisco, Calif., USA, September 1-5 (2004), U.S. Pat. No. 6,454,759). In some embodiments, such ports may be associated with one or more osmotic motors 156. In some embodiments, one or more ports may be opened and/or closed to regulate entry of fluid into one or more chambers of an osmotic motor 156 to control the operation of the motor 156. For example, in some embodiments, one or more ports may be opened to allow fluid to enter into one or more chambers of an osmotic motor 156 to facilitate movement of one or more moveable members 158 that facilitate extrusion of one or more agents 162 from the agent delivery device 128. The one or more ports may be maintained in an open position to provide for entry of fluid into one or more chambers of the osmotic motor 156 or the ports may be closed to disallow entry of fluid into one or more chambers of the osmotic motor 156. Accordingly, in some embodiments, one or more signals may be received by one or more agent delivery devices 128 that provide the one or more agent delivery devices 128 with instructions associated with the delivery of one or more agents 162.

### Electromagnetic Energy

Electrical power may be electromagnetically coupled from one or more electromagnetic energy transmitters 172 with one or more electromagnetic receivers (e.g., sensor electromagnetic receiver 118 and/or agent delivery electromagnetic receiver 132). Accordingly, electrical power that is transferred to the one or more electromagnetic receivers may be used to power one or more operably linked sensors 102 and/or agent delivery devices 128. Electromagnetic energy transmitters 172 that may be modified to transmit electrical power to a sensor 102 and/or agent delivery device 128 have been described (e.g., U.S. Pat. No. 5,571,152).

### External Interface

In some embodiments, system 100 may include one or more external interfaces 168. In some embodiments, one or more external interfaces 168 may be configured to transmit one or more external device signals 170. In some embodiments, one or more external interfaces 168 may be configured to transmit one or more external sensor signals 166. In some embodiments, one or more external interfaces 168 may be configured to receive one or more external device signals 170. In some embodiments, one or more external interfaces 168 may be configured to receive one or more external sensor signals 166. In some embodiments, one or more external interfaces 168 may be configured to transmit electromagnetic energy 164.

Numerous types of electromagnetic energy transmitters 172 may be associated with one or more external interfaces 168. Methods to construct electromagnetic energy transmitters 172 have been described (e.g., U.S. Pat. No. 5,571,152). Briefly, in some embodiments, the electromagnetic energy transmitter 172 may include a ferrite core around which is wrapped an electrical conductor. Other types of material having high magnetic permeability and relatively low magnetic hysteresis may be used for the core. Insulating tape may be wrapped around the electrical conductor, or the electromagnetic energy transmitter 172 may be dipped in a resin to form a coating that stabilizes and fixes the electrical conductor on the core. A return lead from one end of the electrical conductor may include one of two leads that are coupled to an AC power supply.

Numerous types of recording units 178 may be associated with one or more external interfaces 168. Examples of such recording units 178 include, but are not limited to, devices that utilize many types of memory, optical disks, magnetic disks, magnetic tape, and the like. In some embodiments, one or more recording units 178 provide for user interaction.

Numerous types of user interfaces 176 may be associated with one or more external interfaces 168. A user may interact with one or more external interfaces 168 through use of numerous technologies. For example, user interaction can occur through use of hardwired methods, such as through use of a keyboard, use of wireless methods, use of the internet, and the like.

Numerous types of display units 174 may be associated with one or more external interfaces 168. Examples of such display units 174 include, but are not limited to, passive displays, active displays, light emitting diodes, liquid crystal displays, and the like.

An external interface 168 may include one or more external transmitters 180. Numerous types of external transmitters 180 may be used in association with an external interface 168. Examples of such external transmitters 180 include, but are not limited to, transmitters that transmit one or more acoustic signals, optical signals, radio signals, wireless signals, hardwired signals, infrared signals, ultrasonic signals, and the like (e.g., U.S. Pat. Nos. RE39,785; 7,260,768; 7,260,764; 7,260,402; 7,257,327; 7,215,887; 7,218,900; herein incorporated by reference). In some embodiments, one or more external transmitters 180 may transmit one or more signals that are encrypted. Numerous types of transmitters are known and have been described (e.g., U.S. Pat. Nos. and Published U.S. Patent Applications: 7,236,595; 7,260,155; 7,227,956; US2006/0280307; herein incorporated by reference).

An external interface 168 may include one or more external receivers 182. Numerous types of external receivers 182 may be used in association an external interface 168. Examples of such external receivers 182 include, but are not limited to, receivers that receive one or more acoustic signals, optical signals, radio signals, wireless signals, hardwired signals, infrared signals, ultrasonic signals, and the like. Such receivers are known and have been described (e.g., U.S. Pat. Nos. RE39,785; 7,218,900; 7,254,160; 7,245,894; 7,206,605; herein incorporated by reference).

### Agent

Numerous types of agents 162 may be used within system 100. Examples of such agents 162 include, but are not limited to, pharmaceutical agents, hormones, cytokines, and the like. Examples of pharmaceutical agents include, but are not limited to, ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anticholesteremics, anticholinergics, anticoagulants, anticonvulsants, antidepressants, antidiabetics, antidiarrheals, antidiuretics, antidyskinetics, antieczematics, antiemetics, antiestrogens, antifibrotics, antifungals, antiglaucoma agents, antigonadotropins, antigout agents, antihemophilic factors, antihemorrhagics, antihistaminics, antihypercholesterolemics, antihyperlipidemics, antihyperparathyroids, antihyperphosphatemics, antihypertensives, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives, antiulceratives, antiurolithics, antivenins, antivirals, anxiolytic agents, aromatase inhibitors, atriopeptidase inhibitors, benzodiazepine antagonists, beta-blockers, bone resorption inhibitors, bradycardic agents, bradykinin antagonists, bronchodilators, calcium channel blockers, calcium regulators, carbonic anhydrase inhibitors, cardiac depressants, cardioprotective agents, cardiotonics, CCK antagonists, cholelitholytic agents, choleretics, cholinergics, cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, human immunodeficiency virus fusion inhibitors, human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, xanthine oxidase inhibitors, nitric oxide, nitric oxide donors, or substantially any combination thereof.

Examples of hormones include, but are not limited to, estrogen, glucagon-like peptides, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, renin, enkephalin, or substantially any combination thereof.

Examples of cytokines include, but are not limited to, bone morphogenic proteins, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factors, interferon, leptin, macrophage stimulating protein, nerve growth factor, neurotrophin 3, neurotrophin 4, oncostatin M, or substantially any combination thereof.

Following are a series of flowcharts depicting implementations. For ease of understanding, the flowcharts are organized such that the initial flowcharts present implementations via an example implementation and thereafter the following flowcharts present alternate implementations and/or expansions of the initial flowchart(s) as either sub-component operations or additional component operations building on one or more earlier-presented flowcharts. Those having skill in the art will appreciate that the style of presentation utilized herein (e.g., beginning with a presentation of a flowchart(s) presenting an example implementation and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

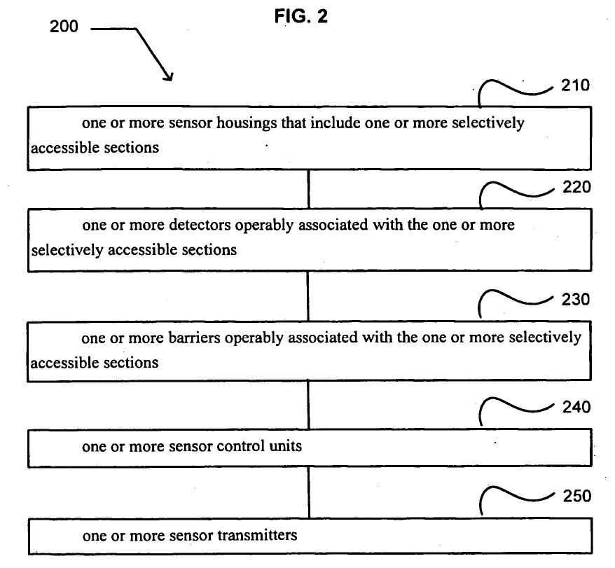

FIG. 2 illustrates embodiment 200 of sensor 102 within system 100. In FIG. 2, discussion and explanation may be provided with respect to the above-described example of FIG. 1, and/or with respect to other examples and contexts. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The embodiment 200 may include module 210 that includes one or more sensor housings that include one or more selectively accessible sections. In some embodiments, a sensor 102 may include one or more sensor housings 184 that include one or more selectively accessible sections 112. Numerous types of material may be used to fabricate one or more sensor housings 184. Examples of such materials include, but are not limited to, metals, metal alloys, plastics, ceramics, polymeric materials, and the like. In some embodiments, one or more sensor housings 184 may include one or more types of materials. For example, in some embodiments, one or more sensor housings 184 may be made of metal and then coated with a polymeric coating. Accordingly, sensor housings 184 may be made of numerous materials and combinations of materials. A sensor 102 may include one or more sensor housings 184 that include one or more selectively accessible sections 112. In some embodiments, a sensor 102 may include one or more sensor housings 184 that include one or more selectively accessible sections 112 that contain one or more detectors 114 that are configured to detect the same type of analyte. In some embodiments, a sensor 102 may include one or more sensor housings 184 that include one or more selectively accessible sections 112 that contain one or more detectors 114 that are configured to detect different types of analytes. In some embodiments, a sensor 102 may include one or more sensor housings 184 that include circuitry for association of one or more sensor receivers 124 with the sensor 102. In some embodiments, a sensor 102 may include one or more sensor housings 184 that include circuitry for association of one or more sensor transmitters 126 with the sensor 102. In some embodiments, a sensor 102 may include one or more sensor housings 184 that include circuitry for association of one or more detectors 114 with the sensor 102. In some embodiments, a sensor 102 may include one or more sensor housings 184 that include circuitry for association of one or more sensor control units 104 with the sensor 102. In some embodiments, a sensor 102 may include one or more sensor housings 184 that include circuitry for association of one or more sensor electromagnetic receivers 118 with the sensor 102. In some embodiments, a sensor 102 may include one or more sensor housings 184 that include circuitry for association of one or more sensor capacitors 122 with the sensor 102. In some embodiments, a sensor 102 may include one or more sensor housings 184 that include circuitry for association of one or more sensor batteries 120 with the sensor 102. In some embodiments, a sensor 102 may include one or more sensor housings 184 that include circuitry for association of one or more analyte detection processors 106 with the sensor 102. In some embodiments, a sensor 102 may include one or more sensor housings 184 that include circuitry for association of one or more sensor receivers 124, sensor transmitters 126, sensor electromagnetic receivers 118, sensor capacitors 122, sensor control units 104, analyte detection processors 106, memories, detectors 114, or substantially any combination thereof with the sensor 102. In some embodiments, a sensor 102 may include one or more sensor housings 184 that include selectively accessible sections 112 that are associated with one or more barriers 186. Numerous types of barriers 186 may be associated with the one or more selectively accessible sections 112. Examples of such barriers 186 include, but are not limited to, metals, shape memory polymers, mechanical doors, electromagnetic valves, and combinations thereof. In some embodiments, a sensor housing 184 may include the same type of barrier 186. In some embodiments, a sensor housing 184 may include different types of barriers 186.

The embodiment 200 may include module 220 that includes one or more detectors operably associated with the one or more selectively accessible sections. In some embodiments, a sensor 102 may include one or more detectors 114 operably associated with the one or more selectively accessible sections 112. Numerous types of detectors 114 may be associated with a sensor 102. Examples of such detectors 114 include, but are not limited to, osmotic detectors 114, micro-electro-mechanical system detectors 114, cantilever detectors 114, electrochemical detectors 114, surface plasmon resonance detectors 114, electrodes, detectors 114 configured to detect nitric oxide, detectors 114 configured to detect glucose, detectors 114 configured to detect one or more hormones, detectors 114 configured to detect one or more pharmaceutical agents, detectors 114 configured to detect one or more cytokines, detectors 114 configured to detect one or more pathogen indicators, or substantially any combination thereof. In some embodiments, a sensor 102 may include one type of detector 114. In some embodiments, a sensor may include one or more types of detectors 114.

The embodiment 200 may include module 230 that includes one or more barriers operably associated with the one or more selectively accessible sections. In some embodiments, a sensor 102 may include one or more barriers 186 operably associated with the one or more selectively accessible sections 112. In some embodiments, one or more selectively accessible sections 112 may be operably associated with one or more barriers 186 that sequester a portion of the selectively accessible section 112 from an outside environment. For example, in some embodiments, a selectively accessible section 112 may be sequestered from the outside environment by an impermeable metal foil barrier 186. In some embodiments, one or more selectively accessible sections 112 may be operably associated with one or more barriers 186 that selectively sequester the selectively accessible section 112 from an outside environment. For example, in some embodiments, a selectively accessible section 112 may be selectively sequestered from the outside environment by a selectively permeable metal foil barrier 186 (e.g., a perforated gold foil that is selectively permeable based on molecular weight).

The embodiment 200 may include module 240 that includes one or more sensor control units. In some embodiments, a sensor 102 may include one or more sensor control units 104. In some embodiments, a sensor 102 may include one or more sensor control units 104 that include one or more analyte detection processors 106. In some embodiments, a sensor 102 may include one or more sensor control units 104 that include one or more analyte detection memories 110. In some embodiments, a sensor 102 may include one or more sensor control units 104 that include analyte detection logic 108. In some embodiments, a sensor 102 may include one or more sensor control units 104 that are configured to facilitate unmasking of one or more selectively accessible sections 112 associated with the sensor 102. In some embodiments, a sensor 102 may include one or more sensor control units 104 that are configured to control the operation of one or more sensor transmitters 126. In some embodiments, a sensor 102 may include one or more sensor control units 104 that are configured to control the operation of one or more sensor receivers 124. In some embodiments, a sensor 102 may include one or more sensor control units 104 that are configured to control the operation of one or more sensor power sources 116. In some embodiments, a sensor 102 may include one or more sensor control units 104 that are configured to control the operation of one or more detectors 114.

The embodiment 200 may include module 250 that includes one or more sensor transmitters. In some embodiments, a sensor 102 may include one or more sensor transmitters 126. A sensor 102 may include one or more sensor transmitters 126 that are configured to transmit numerous types of signals. Examples of such signals include, but are not limited to, acoustic signals, electromagnetic signals, optical signals, infrared signals, radio signals, radio frequency signals, microwave signals, ultrasonic signals, or substantially any combination thereof. In some embodiments, one or more sensor transmitters 126 may be configured to transmit one or more signals according to one or more schedules. In some embodiments, one or more schedules may be time schedules. In some embodiments, one or more schedules may be agent administration schedules. In some embodiments, one or more sensor transmitters 126 may be configured to transmit one or more signals in response to detection of one or more analytes. For example, in some embodiments, one or more sensor transmitters 126 may transmit one or more signals if one or more analytes are detected. In some embodiments, one or more sensor transmitters 126 may transmit one or more signals if one or more analytes are not detected. In some embodiments, one or more sensor transmitters 126 may transmit one or more signals in response to one or more queries. For example, in some embodiments, one or more sensor transmitters 126 may transmit one or more signals in response to a query from one or more agent delivery devices 128.

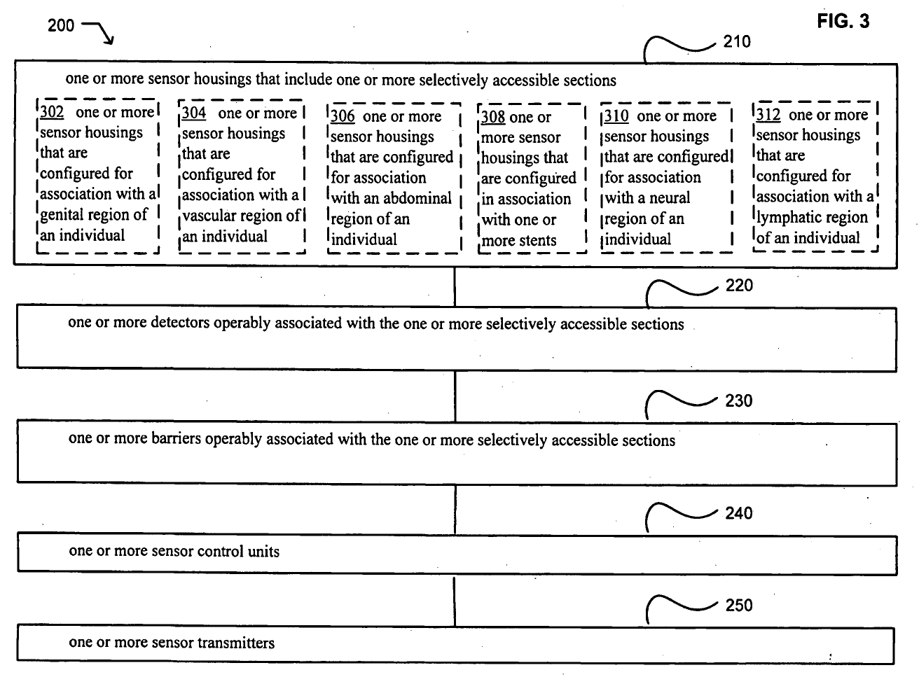

FIG. 3 illustrates alternative embodiments of embodiment 200 of sensor 102 within system 100 of FIG. 2. FIG. 3 illustrates example embodiments of module 210. Additional embodiments may include an embodiment 302, an embodiment 304, an embodiment 306, an embodiment 308, an embodiment 310, and/or an embodiment 312.

At embodiment 302, module 210 may include one or more sensor housings that are configured for association with a genital region of an individual. In some embodiments, one or more sensor housings 184 may include one or more sensor housings 184 that are configured for association with a genital region of an individual. One or more sensors 102 may include one or more sensor housings 184 that are configured to associate with numerous types of detectors 114. In some embodiments, one or more sensors 102 may include one or more sensor housings 184 that are configured for association with male genitalia. For example, in some embodiments, one or more sensor housings 184 may be configured for association with the penile corpus cavernosum of an animal. In some embodiments, one or more sensor housings 184 may be configured for implantation within the penile corpus cavernosum of an animal. For example, in some embodiments, one or more sensors 102 may be associated with a stent (e.g., U.S. Pat. Nos. 6,442,413 and 5,514,176). In some embodiments, such stents may be configured for implantation within the corpus cavemosum of an animal. In some embodiments, one more sensor housings 184 may be associated with one or more detectors 114 that are configured to detect nitric oxide. In some embodiments, one or more sensor housings 184 may be associated with one or more detectors 114 that are configured to detect one or more nitric oxide donors. In some embodiments, one or more sensors 102 may include one or more sensor housings 184 that are configured for implantation into the scrotum of an animal. For example, in some embodiments, one or more sensors 102 may include a sensor housing 184 that is configured as a prosthetic testicle. In some embodiments, one or more sensors 102 may include one or more sensor housings 184 that are configured for association with female genitalia. For example, in some embodiments, one or more sensors 102 may include one or more sensor housings 184 that are configured for intrauterine placement (e.g., an intrauterine device). In some embodiments, one or more sensors 102 may include one or more sensor housings 184 that are configured for placement with a fallopian tube of a female. In some embodiments, such sensors 102 may include one or more sensor housings 184 that are configured as a stent.

In some embodiments, one or more sensors 102 that are configured for association with a genital region of an individual may include one or more detectors 114 that are configured to detect one or more disease markers. For example, in some embodiments, one or more sensors 102 that are configured for association with a genital region of an individual may include one or more detectors 114 that are configured to detect one or more cancer markers. In some embodiments, one or more sensors 102 that are configured for association with a genital region of an individual may include one or more detectors 114 that are configured to detect ovarian cancer (e.g., CA 125, CA 15.3). In some embodiments, one or more sensors 102 that are configured for association with a genital region of an individual may include one or more detectors 114 that are configured to detect uterine cancer (e.g., CA 125). In some embodiments, one or more sensors 102 that are configured for association with a genital region of an individual may include one or more detectors 114 that are configured to detect cervical cancer (e.g., telomerase activity (Reddy et al., Int. Jour. Gyn. Cancer, 11:100-106 (2001), ether a go-go potassium channels (Farias et al., Cancer Research, 64:6996-7001 (2004), U.S. Pat. No. 7,125,663; U.S. Published Patent Application No.: 20060160069). In some embodiments, one or more sensors 102 that are configured for association with a genital region of an individual may include one or more detectors 114 that are configured to detect testicular cancer (e.g., lactate dehydrogenase). Accordingly, one or more sensors 102 may be configured to detect numerous types of tumor markers.

At embodiment 304, module 210 may include one or more sensor housings that are configured for association with a vascular region of an individual. In some embodiments, one or more sensor housings 184 may include one or more sensor housings 184 that are configured for association with a vascular region of an individual. In some embodiments, one or more sensor housings 184 may be configured as a stent (e.g., U.S. Pat. Nos. 6,442,413; 7,236,821; and 5,514,176). In some embodiments, one or more sensors 102 may be configured for association with another implantable device. For example, in some embodiments, one or more sensors 102 may be configured for association with one or more pacemaker leads. One or more sensor housings 184 that are configured for implantation in a vascular region of an individual may be configured to associate with numerous types of detectors 114. In some embodiments, such detectors 114 may include one or more detectors 114 that are configured to detect one or more disease markers (e.g., cancer, hypertension, hypotension, hypercholesterolemia, pathogen infection, hormone imbalance, etc.). In some embodiments, such detectors 114 may include one or more detectors 114 that are configured to detect one or more cancer markers (e.g., cancer antigen 125, cancer antigen 15.3, cancer antigen 27.29, carcinoembryonic antigen, carbohydrate antigen 19-9, neuron-specific enolase, carcinoembryonic antigen, lactate dehydrogenase, HER2, prostate-specific antigen, acid phosphatase, alpha-fetoprotein). Accordingly, in some embodiments, systems may be constructed that include one or more sensors 102 that include cancer marker detectors that are paired with one or more agent delivery devices 128 that administer one or more chemotherapy agents to an individual. In some embodiments, such detectors 114 may include one or more detectors 114 that are configured to detect blood pressure (e.g., U.S. Pat. No. 6,713,828). Accordingly, in some embodiments, systems may be constructed that include one or more sensors 102 that include blood pressure detectors 114 that are paired with one or more agent delivery devices 128 that administer one or more nitric oxide donors to an individual. In some embodiments, such systems may be used to treat hypertension and/or stroke (e.g., U.S. Pat. Nos. 5,039,705 and 5,385,940). In some embodiments, such detectors 114 may include one or more detectors 114 that are configured to detect changes in blood pressure. In some embodiments, such detectors 114 may include one or more detectors 114 that are configured to detect blood sugar concentration. In some embodiments, such detectors 114 may include one or more detectors 114 that are configured to detect blood glucose concentration (e.g., U.S. Pat. Nos. 7,110,803; 6,210,326; 6,546,268; 6,965,791; 4,703,756; 6,268,161). In some embodiments, such detectors 114 may include one or more detectors 114 that are configured to detect one or more lipids. In some embodiments, such detectors 114 may include one or more detectors 114 that are configured to detect one or more pathogen indicators. In some embodiments, such detectors 114 may include one or more detectors 114 that are configured to detect one or more nitric oxide donors. In some embodiments, such detectors 114 may include one or more detectors 114 that are configured to detect one or more nitric oxide synthases. In some embodiments, such detectors 114 may include one or more detectors 114 that are configured to detect nitric oxide.

Accordingly, in some embodiments, systems may be constructed that include one or more sensors 102 that include nitric oxide detectors 114 that are paired with one or more agent delivery devices 128 that administer one or more agents 162 that inhibit nitric oxide production (e.g., one or more substituted arginines (e.g., U.S. Pat. No. 5,028,627)) to an individual. In some embodiments, such systems may be used to treat hypotension, such as hypotension caused by septic shock (e.g., U.S. Pat. No. 5,028,627).

At embodiment 306, module 210 may include one or more sensor housings that are configured for association with an abdominal region of an individual. In some embodiments, one or more sensor housings 184 may include one or more sensor housings 184 that are configured for association with an abdominal region of an individual. In some embodiments, one or more sensor housings 184 may be configured for implantation into an abdominal region of an individual. In some embodiments, one or more sensor housings 184 may be configured for passage through the gastrointestinal tract of an individual. In some embodiments, one or more sensor housings 184 may be configured for implantation into a prostate gland (e.g., U.S. Published Patent Application No.: 20050096709). Accordingly, in some embodiments, one or more sensors 102 may be used to monitor prostate specific antigen concentration in prostate tissue. In some embodiments, one or more sensors 102 may be configured to detect an agent 162 associated with treatment of prostate cancer. For example, in some embodiments, a sensor 102 may be configured to detect leuprolide acetate concentration. In some embodiments, one or more sensors 102 that are configured to detect leuprolide acetate may be operably coupled with one or more agent delivery devices 128 that are configured to administer leuprolide acetate to an individual. Accordingly, in some embodiments, one or more sensors 102 and one or more agent delivery devices 128 can be configured to maintain the concentration of leuprolide acetate within an individual at a setpoint and/or within a range of concentrations. One or more sensor housings 184 may be configured in numerous ways to facilitate association with an abdominal region of an individual.

At embodiment 308, module 210 may include one or more sensor housings that are configured in association with one or more stents. In some embodiments, one or more sensor housings 184 may include one or more sensor housings 184 that are configured for association with one or more stents. In some embodiments, one or more sensor housings 184 may be configured as a stent. For example, in some embodiments, a stent may include one or more selectively accessible sections 112 that are integrated into the structure of the stent. Accordingly, in some embodiments, one or more barriers 186 may be operably associated with the one or more selectively accessible sections 112 that are integral to the stent. In some embodiments, one or more sensor housings 184 may be configured for attachment to a stent. For example, in some embodiments, one or more sensor housings 184 may be configured for attachment to the interior surface of a stent. Accordingly, in some embodiments, a sensor 102 may be configured so that one or more detectors 114 are oriented to detect one or more analytes within the interior of the stent upon unmasking of one or more selectively accessible sections 112 that enclose the one or more detectors 114. In some embodiments, one or more sensor housings 184 may be configured for attachment to the exterior surface of a stent. Accordingly, in some embodiments, such sensor housings 184 may be configured so that one or more detectors 114 are oriented to detect one or more analytes within an area between the stent and a luminal wall to which the stent is affixed. In some embodiments, a sensor 102 may include one or more sensor housings 184 that may be configured to include one or more nitric oxide detectors 114 that are oriented to detect nitric oxide between an outside surface of a stent and an inside surface of a lumen within which the stent is implanted. Accordingly, in some embodiments, systems may be constructed that include one or more such sensors 102 that are operably coupled to one or more agent delivery devices 128 that administer nitric oxide, and/or one or more nitric oxide donors, to the implantation site of the stent. Housings that are associated with a stent may be configured in numerous ways. For example, such sensor housings 184 may be configured for association with stents that are configured for implantation within numerous types of bodily structures. Examples of such bodily structures include, but are not limited to, arteries, veins, capillaries, a urethra, a fallopian tube, an intestine, an esophagus, a trachea, a corpus cavernosum, and the like. Accordingly, sensors 102 may be incorporated into numerous types of stents and stent-like structures.

At embodiment 310, module 210 may include one or more sensor housings that are configured for association with a neural region of an individual. In some embodiments, one or more sensor housings 184 may include one or more sensor housings 184 that are configured for association with a neural region of an individual. In some embodiments, one or more sensor housings 184 may be configured to associate with the central nervous system of an individual. In some embodiments, one or more sensor housings 184 may be configured to associate with the peripheral nervous system of an individual. In some embodiments, one or more sensor housings 184 may be configured for implantation into the brain of an individual. In some embodiments, one or more sensor housings 184 may be configured for detection of brain activity. For example, in some embodiments, one or more sensor housings 184 may be configured for cranial implantation to facilitate detection of an epileptic seizure. Accordingly, in some embodiments, one or more sensor housings 184 may be configured to facilitate detection of an epileptic seizure and transmission of one or more internal signals 160 that include information with regard to antiepileptic drugs (e.g., gabapentin, lamotrigine, felbamate, topiramate, fosphenyloin) for administration to the individual. In some embodiments, one or more sensor housings 184 may be configured to facilitate detection of one or more agents 162 that are associated with the treatment of depression. Accordingly, one or more sensor housings 184 may configured in numerous ways.

At embodiment 312, module 210 may include one or more sensor housings that are configured for association with a lymphatic region of an individual. In some embodiments, one or more sensor housings 184 may include one or more sensor housings 184 that are configured for association with a lymphatic region of an individual. In some embodiments, one or more sensor housings 184 may be configured to facilitate detection of one or more components of the lymphatic system (e.g., cells, fatty acids, chyle). In some embodiments, one or more sensor housings 184 may be configured to facilitate detection of one or more cancer cells.

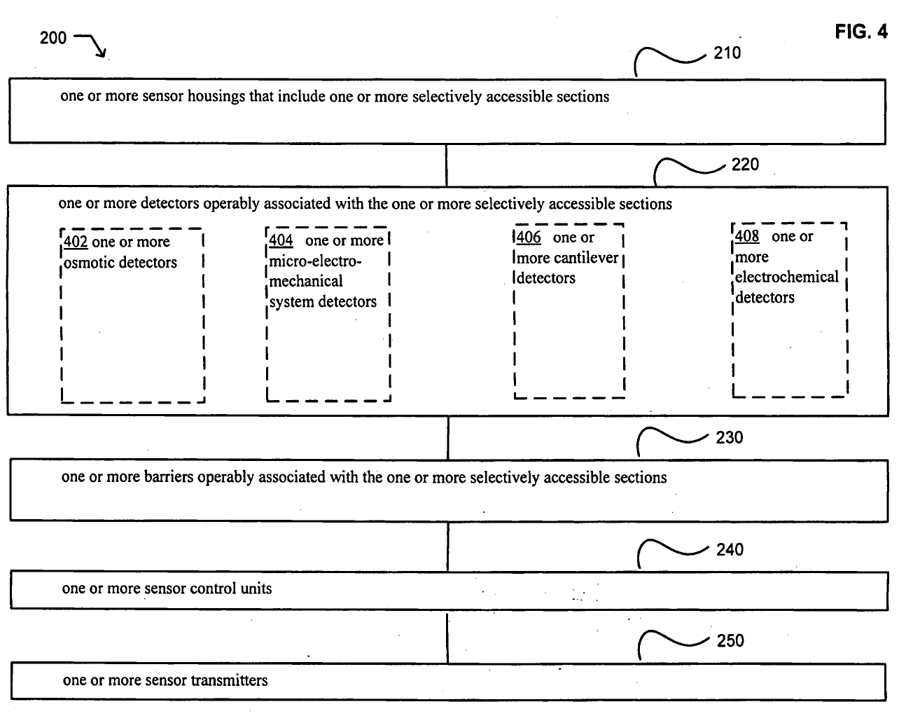

FIG. 4 illustrates alternative embodiments of embodiment 200 of sensor 102 within system 100 of FIG. 2. FIG. 4 illustrates example embodiments of module 220. Additional embodiments may include an embodiment 402, an embodiment 404, an embodiment 406, and/or an embodiment 408.

At embodiment 402, module 220 may include one or more osmotic detectors. In some embodiments, one or more detectors 114 operably associated with the one or more selectively accessible sections 112 may include one or more osmotic detectors 114. In some embodiments, osmotic detectors 114 may be configured to detect one or more analytes through detecting a change in the osmotic pressure associated with the detector 114 that is due to the presence or absence of an analyte. In some embodiments, an osmotic detector 114 may be configured as a hydrogel (e.g., U.S. Pat. Nos. 6,514,689; 6,835,553; and 6,268,161). In some embodiments, the hydrogel may include an immobilized analyte binder and an immobilized analyte. The immobilized analyte competitively binds with free analyte to the analyte binder, thus changing the number of crosslinks in the hydrogel, which changes hydrogel swelling tendency (and thus the osmotic pressure) in its confined space in proportion to the concentration of free analyte concentration. Accordingly, numerous types of analytes and analyte binders may be included within one or more osmotic detectors. Numerous analyte binders may be immobilized on the ZnO nanotips. Examples of such analyte binders include, but are not limited to, chelating agents, antibodies (monoclonal, polyclonal, single-chain, antibody fragments, etc.), aptamers (e.g., nucleic acid, peptide), receptors (T-cell receptors, hormone receptors, cytokine receptors, sugar receptors, etc.), binding proteins (nucleic acid binding proteins, maltose binding protein, calcium binding protein, enzymes, etc.), nucleic acids, microorganisms, tissues, organelles, whole cells, and the like.

At embodiment 404, module 220 may include one or more micro-electro-mechanical system detectors. In some embodiments, one or more detectors 114 operably associated with the one or more selectively accessible sections 112 may include one or more micro-electro-mechanical system detectors 114. One or more sensors 102 may include numerous types of micro-electro-mechanical system detectors 114. For example, in some embodiments, one or more sensors 102 may include one or more detectors 114 that include a ZnO nanotip bulk acoustic wave resonator detector 114 (e.g., U.S. Published Patent Nos.: 20070210349 and 20050116263). In some embodiments, such a detector 114 may include a piezoelectric layer; a conductive film that serves as a bottom electrode that is deposited and patterned beneath the piezoelectric layer; a metal electrode that serves as a top electrode that is deposited and patterned on the piezoelectric layer; and ZnO nanotips that are deposited and patterned on the top surface. In some embodiments, binding of one or more analytes with analyte binders that are immobilized on the ZnO nanotips causes mass loading on the detector which causes a change in the resonance frequency of the detector. In some embodiments, such a detector 114 may be configured as a quartz crystal microbalance. In some embodiments, such a detector 114 may be configured as a thin film bulk acoustic wave resonator. Numerous analyte binders may be immobilized on the ZnO nanotips. Examples of such analyte binders include, but are not limited to, chelating agents, antibodies (monoclonal, polyclonal, single-chain, antibody fragments, etc.), aptamers (e.g., nucleic acid, peptide), receptors (T-cell receptors, hormone receptors, cytokine receptors, sugar receptors, etc.), binding proteins (nucleic acid binding proteins, maltose binding protein, calcium binding protein, enzymes, etc.), nucleic acids, microorganisms, tissues, organelles, whole cells, and the like.

In some embodiments, one or more micro-electro-mechanical system detectors 114 may be configured to detect pressure (e.g., U.S. Pat. No. 6,939,299). In some embodiments, a micro-electro-mechanical system detector 114 may include a pressure sensing capacitor and an inductor. The capacitor and the inductor may be integrated within a micromachined chip, which forms an inductor/capacitor resonant circuit (or resonant LC circuit) that is characterized by a resonant frequency. In some embodiments, the inductor may be configured as a coil. The inductor and the first capacitor plate may be associated with a membrane, such as a glass substrate, sealed and electrically isolated inside the detector. The detector 114 may further include a deformable membrane that is bonded to the glass substrate and associated with the second capacitor plate. Fluid pressure deflects the membrane and the second capacitor plate. These pressure-induced motions of the membrane change the capacitance value and thereby change the resonant frequency of the LC circuit. An increase in pressure causes an increase in capacitance, which causes a decrease in resonant frequency. Such a pressure sensor does not require an internal or external energy source. Accordingly, sensors 102 may include one or more micro-electro-mechanical system detectors 114 that are configurd in numerous ways.

At embodiment 406, module 220 may include one or more cantilever detectors. In some embodiments, one or more detectors 114 operably associated with the one or more selectively accessible sections 112 may include one or more cantilever detectors 114. Numerous types of cantilever detectors 114 may be associated with one or more sensors 102 (e.g., U.S. Pat. Nos. 7,168,294; 7,288,404; and 6,823,717). In some embodiments, a cantilever detector 114 may include a deflectable arm of a microcantilever that is at least partially embedded within a chamber of the detector 114. Entry of an analyte into the chamber will cause the chamber to undergo a volumetric expansion or contraction in the presence of the analyte. In some embodiments, such an expansion or contraction may occur through absorption of an analyte. The volumetric change of the chamber causes the deflectable arm to deflect. The deflectable arm includes at least one measurable physical property which changes when the arm deflects. Changes in the physical property may be used to determine the presence and amount of analyte present within the chamber. In some embodiments, one or more piezoelectric detectors 114 may be associated with one or more cantilever detectors 114. In some embodiments, one or more analyte binders may be associated with a microcantilever arm. Examples of such analyte binders include, but are not limited to, chelating agents, antibodies (monoclonal, polyclonal, single-chain, antibody fragments, etc.), aptamers (e.g., nucleic acid, peptide), receptors (T-cell receptors, hormone receptors, cytokine receptors, sugar receptors, etc.), binding proteins (nucleic acid binding proteins, maltose binding protein, calcium binding protein, etc.), nucleic acids, and the like. Cantilever detectors 114 may be configured in numerous ways.

At embodiment 408, module 220 may include one or more electrochemical detectors. In some embodiments, one or more detectors 114 operably associated with the one or more selectively accessible sections 112 may include one or more electrochemical detectors 114. Electrochemical detectors 102 may be configured in numerous ways (e.g., U.S. Pat. Nos. 6,436,699 and 4,935,345). In some embodiments, one or more electrochemical detectors 114 may be configured to detect nitric oxide. For example, in some embodiments, a nitric oxide detector 114 may include an electropolymerized film of o-aminobenzaldehyde-ethylene-diamine nickel and tetrafluoroethylene-perfluoro-3,6-dioxa-4-methyl-7-octenesulfonic acid copolymer (e.g., Tu et al., Electroanalysis, 11:70-74 (1999)). In some embodiments, one or more electrochemical detectors 114 may include an analyte binder that is bound to an electrode and an analyte/enzyme conjugate that is bound to the analyte binder as part of a catalytic electrical circuit. Displacement of the conjugate by an analyte will cause a measurable change in current (U.S. Pat. No. 5,391,272). In some embodiments, one or more electrochemical detectors 114 may include one or more chemically-sensitive resistors that are configured to interact with an analyte of interest. In such embodiments, interaction of an analyte with the one or more chemically-sensitive resistors provides a resistance fingerprint. This fingerprint can be associated with a library to determine the analyte's activity (U.S. Pat. No. 6,350,369). In some embodiments, one or more electrochemical detectors 114 may include a working electrode and a counter electrode. In some embodiments, the detector 114 measures the concentration of an analyte by discharging an amount of charge into a sample, determining the time needed to discharge the charge, and determining the current used to electrolyze a portion of the analyte using the amount of charge and the amount of time (U.S. Pat. No. 6,749,740).

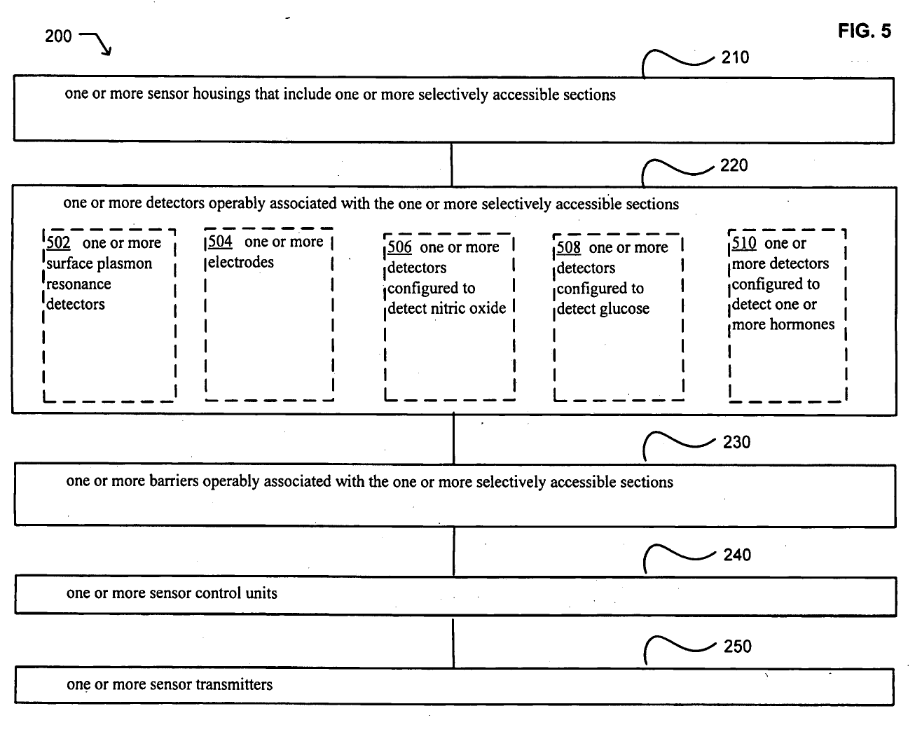

FIG. 5 illustrates alternative embodiments of embodiment 200 of sensor 102 within system 100 of FIG. 2. FIG. 5 illustrates example embodiments of module 220. Additional embodiments may include an embodiment 502, an embodiment 504, an embodiment 506, an embodiment 508, and/or an embodiment 510.

At embodiment 502, module 220 may include one or more surface plasmon resonance detectors. In some embodiments, one or more detectors 114 operably associated with the one or more selectively accessible sections 112 may include one or more surface plasmon resonance detectors 114. A sensor 102 may be operably associated with plasmon resonance detectors 114 that are configured in numerous ways (e.g., U.S. Pat. Nos. 5,492,840; 6,330,464; 6,862,465). In some embodiments, a detector 114 may include one or more surface plasmon resonance detectors 114 that include a dielectric substrate which is coated with a metal film having two or more detection areas that are arranged to facilitate passage of a liquid stream. In some embodiments, the detection areas may include a layer of an organic polymer or hydrogel over the metal film with which one or more analyte binders are associated (e.g., U.S. Pat. No. 5,492,840). Surface plasmon resonance detectors 114 may be associated with numerous types of analyte binders. Examples of such analyte binders include, but are not limited to, chelating agents, antibodies (monoclonal, polyclonal, single-chain, antibody fragments, etc.), aptamers (e.g., nucleic acid, peptide), receptors (T-cell receptors, hormone receptors, cytokine receptors, sugar receptors, etc.), binding proteins (nucleic acid binding proteins, maltose binding protein, calcium binding protein, etc.), nucleic acids, and the like.

At embodiment 504, module 220 may include one or more electrodes. In some embodiments, one or more detectors 114 operably associated with the one or more selectively accessible sections 112 may include one or more electrodes. A sensor 102 may be operably coupled to one or more electrodes that are configured in numerous ways (e.g., U.S. Pat. Nos. 6,329,161 and 7,025,734). In some embodiments, one or more electrodes may be configured to detect nitric oxide (e.g., U.S. Pat. Nos. 6,280,604; 5,466,350; 5,603,820; and 5,980,705). In some embodiments, one or more electrodes may be configured to detect pH (e.g., U.S. Pat. No. 6,689,056). In some embodiments, one or more electrodes may be configured to detect glucose (e.g., U.S. Pat. Nos. 6,162,611 and 5,387,327). Accordingly, one or more sensors 102 may include one or more detectors 114 that include electrodes that are configured in numerous ways.

At embodiment 506, module 220 may include one or more detectors configured to detect nitric oxide. In some embodiments, one or more detectors 114 operably associated with the one or more selectively accessible sections 112 may include one or more detectors 114 configured to detect nitric oxide. A sensor 102 may be operably associated with nitric oxide detectors 114 that are configured in numerous ways (e.g., U.S. Pat. Nos. 7,025,734; 7,291,503; 6,287,452; 5,582,170; 5,603,820; 6,280,604; and 6,280,604). In some embodiments, one or more sensors 102 that are operably associated with one or more detectors 114 configured to detect nitric oxide may be associated with one or more stents. Accordingly, in some embodiments, systems may be constructed that include one or more sensors 102 that include one or more nitric oxide detectors 114 that are paired with one or more agent delivery devices 128 that administer nitric oxide to an individual. In some embodiments, such administration may improve stent implantation. In some embodiments, such administration may reduce restenosis (Do et al., Radiology, 230:377-382 (2004) and Vermeersch et al., Arterioscler. Thromb. Vasc. Biol., 21:1604-1609 (2001)).

At embodiment 508, module 220 may include one or more detectors configured to detect glucose. In some embodiments, one or more detectors 114 operably associated with the one or more selectively accessible sections 112 may include one or more detectors 114 configured to detect glucose. A sensor 102 may be operably associated with nitric oxide detectors 114 that are configured in numerous ways (e.g., U.S. Pat. Nos. 7,162,289; 6,210,326; 6,546,268; 6,965,791; 4,703,756; and 7,110,803). Examples of such configurations include, but are not limited to, osmotic detectors 114, electrochemical detectors 114, surface plasmon resonance detectors 114, electrochemical detectors 114, cantilever detectors 114, micro-electro-mechanical system detectors 114, and the like.

At embodiment 510, module 220 may include one or more detectors configured to detect one or more hormones. In some embodiments, one or more detectors 114 operably associated with the one or more selectively accessible sections 112 may include one or more detectors 114 configured to detect one or more hormones. A sensor 102 may include one or more detectors 114 that are configured in numerous ways to facilitate detection of one or more hormones. Examples of such configurations include, but are not limited to, osmotic detectors 114, electrochemical detectors 114, surface plasmon resonance detectors 114, electrochemical detectors 114, cantilever detectors 114, micro-electro-mechanical system detectors 114, and the like.

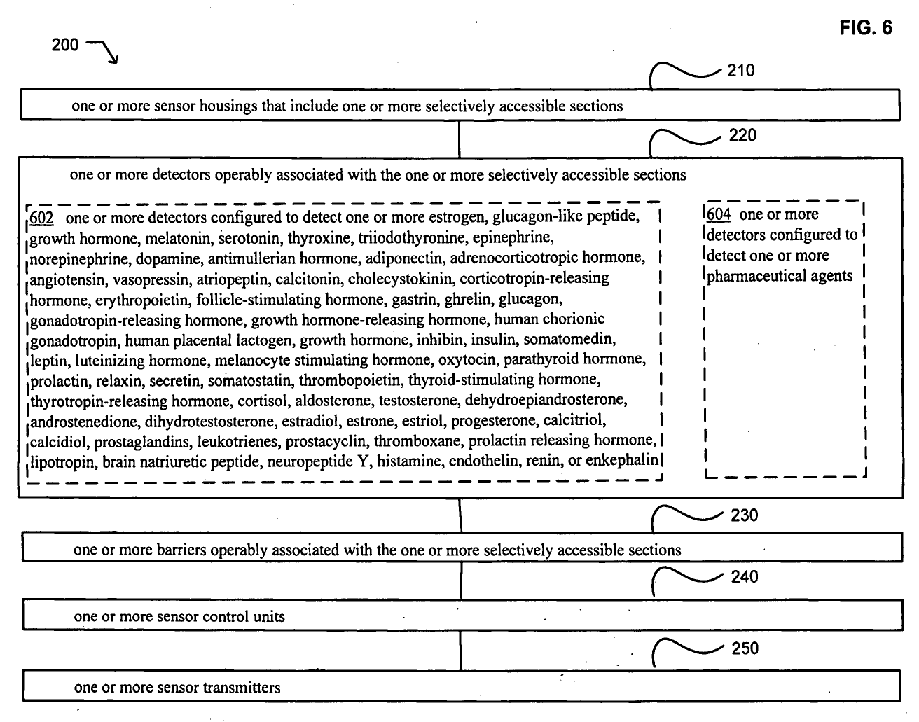

FIG. 6 illustrates alternative embodiments of embodiment 200 of sensor 102 within system 100 of FIG. 2. FIG. 6 illustrates example embodiments of module 220. Additional embodiments may include an embodiment 602, and/or an embodiment 604.

At embodiment 602, module 220 may include one or more detectors configured to detect one or more estrogen, glucagon-like peptide, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, renin, or enkephalin. In some embodiments, one or more detectors 114 operably associated with the one or more selectively accessible sections 112 may include one or more detectors 114 configured to detect one or more estrogen, glucagon-like peptide, growth hormone, melatonin, serotonin, thyroxine, triiodothyronine, epinephrine, norepinephrine, dopamine, antimullerian hormone, adiponectin, adrenocorticotropic hormone, angiotensin, vasopressin, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, somatomedin, leptin, luteinizing hormone, melanocyte stimulating hormone, oxytocin, parathyroid hormone, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, renin, enkephalin, or substantially any combination thereof.

At embodiment 604, module 220 may include one or more detectors configured to detect one or more pharmaceutical agents. In some embodiments, one or more detectors 114 operably associated with the one or more selectively accessible sections 112 may include one or more detectors 114 configured to detect one or more pharmaceutical agents 162. A sensor 102 may include one or more detectors 114 that are configured in numerous ways to facilitate detection of one or more pharmaceutical agents 162. Examples of such configurations include, but are not limited to, osmotic detectors 114, electrochemical detectors 114, surface plasmon resonance detectors 114, electrochemical detectors 114, cantilever detectors 114, micro-electro-mechanical system detectors 114, and the like.

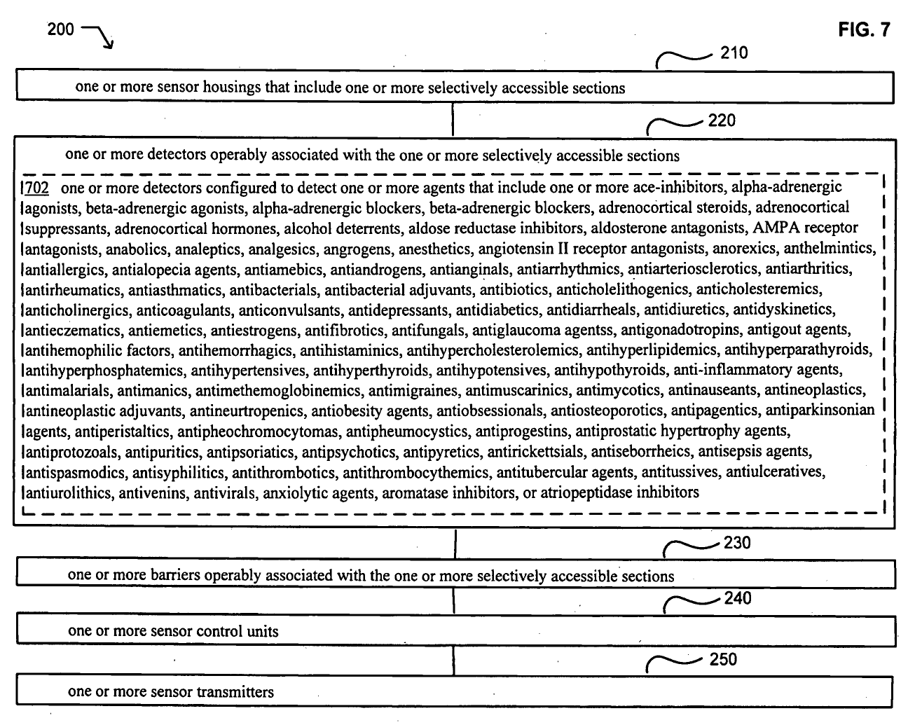

FIG. 7 illustrates alternative embodiments of embodiment 200 of sensor 102 within system 100 of FIG. 2. FIG. 7 illustrates example embodiments of module 220. Additional embodiments may include an embodiment 702.

At embodiment 702, module 220 may include one or more detectors configured to detect one or more agents that include one or more ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anticholesteremics, anticholinergics, anticoagulants, anticonvulsants, antidepressants, antidiabetics, antidiarrheals, antidiuretics, antidyskinetics, antieczematics, antiemetics, antiestrogens, antifibrotics, antifungals, antiglaucoma agentss, antigonadotropins, antigout agents, antihemophilic factors, antihemorrhagics, antihistaminics, antihypercholesterolemics, antihyperlipidemics, antihyperparathyroids, antihyperphosphatemics, antihypertensives, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives, antiulceratives, antiurolithics, antivenins, antivirals, anxiolytic agents, aromatase inhibitors, or atriopeptidase inhibitors. In some embodiments, one or more detectors 114 operably associated with the one or more selectively accessible sections 112 may include one or more detectors 114 configured to detect one or more agents 162 that include one or more ace-inhibitors, alpha-adrenergic agonists, beta-adrenergic agonists, alpha-adrenergic blockers, beta-adrenergic blockers, adrenocortical steroids, adrenocortical suppressants, adrenocortical hormones, alcohol deterrents, aldose reductase inhibitors, aldosterone antagonists, AMPA receptor antagonists, anabolics, analeptics, analgesics, angrogens, anesthetics, angiotensin II receptor antagonists, anorexics, anthelmintics, antiallergics, antialopecia agents, antiamebics, antiandrogens, antianginals, antiarrhythmics, antiarteriosclerotics, antiarthritics, antirheumatics, antiasthmatics, antibacterials, antibacterial adjuvants, antibiotics, anticholelithogenics, anticholesteremics, anticholinergics, anticoagulants, anticonvulsants, antidepressants, antidiabetics, antidiarrheals, antidiuretics, antidyskinetics, antieczematics, antiemetics, antiestrogens, antifibrotics, antifungals, antiglaucoma agentss, antigonadotropins, antigout agents, antihemophilic factors, antihemorrhagics, antihistaminics, antihypercholesterolemics, antihyperlipidemics, antihyperparathyroids, antihyperphosphatemics, antihypertensives, antihyperthyroids, antihypotensives, antihypothyroids, anti-inflammatory agents, antimalarials, antimanics, antimethemoglobinemics, antimigraines, antimuscarinics, antimycotics, antinauseants, antineoplastics, antineoplastic adjuvants, antineurtropenics, antiobesity agents, antiobsessionals, antiosteoporotics, antipagentics, antiparkinsonian agents, antiperistaltics, antipheochromocytomas, antipheumocystics, antiprogestins, antiprostatic hypertrophy agents, antiprotozoals, antipuritics, antipsoriatics, antipsychotics, antipyretics, antirickettsials, antiseborrheics, antisepsis agents, antispasmodics, antisyphilitics, antithrombotics, antithrombocythemics, antitubercular agents, antitussives, antiulceratives, antiurolithics, antivenins, antivirals, anxiolytic agents, aromatase inhibitors, atriopeptidase inhibitors, or substantially any combination thereof.

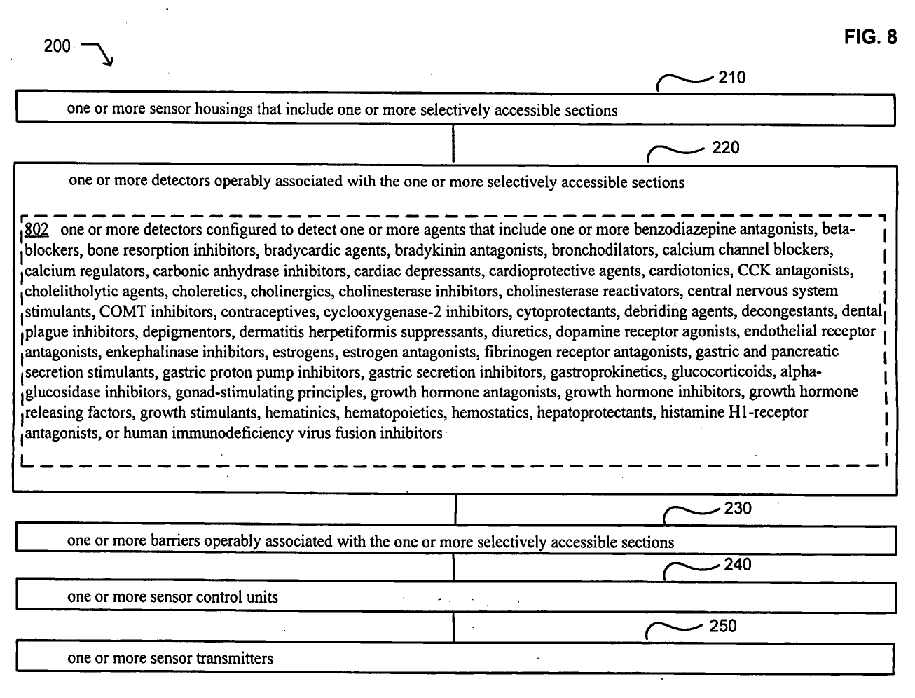

FIG. 8 illustrates alternative embodiments of embodiment 200 of sensor 102 within system 100 of FIG. 2. FIG. 8 illustrates example embodiments of module 220. Additional embodiments may include an embodiment 802.

At embodiment 802, module 220 may include one or more detectors configured to detect one or more agents that include one or more benzodiazepine antagonists, beta-blockers, bone resorption inhibitors, bradycardic agents, bradykinin antagonists, bronchodilators, calcium channel blockers, calcium regulators, carbonic anhydrase inhibitors, cardiac depressants, cardioprotective agents, cardiotonics, CCK antagonists, cholelitholytic agents, choleretics, cholinergics, cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, or human immunodeficiency virus fusion inhibitors. In some embodiments, one or more detectors 114 operably associated with the one or more selectively accessible sections 112 may include one or more detectors 114 configured to detect one or more agents 162 that include one or more benzodiazepine antagonists, beta-blockers, bone resorption inhibitors, bradycardic agents, bradykinin antagonists, bronchodilators, calcium channel blockers, calcium regulators, carbonic anhydrase inhibitors, cardiac depressants, cardioprotective agents, cardiotonics, CCK antagonists, cholelitholytic agents, choleretics, cholinergics, cholinesterase inhibitors, cholinesterase reactivators, central nervous system stimulants, COMT inhibitors, contraceptives, cyclooxygenase-2 inhibitors, cytoprotectants, debriding agents, decongestants, dental plague inhibitors, depigmentors, dermatitis herpetiformis suppressants, diuretics, dopamine receptor agonists, endothelial receptor antagonists, enkephalinase inhibitors, estrogens, estrogen antagonists, fibrinogen receptor antagonists, gastric and pancreatic secretion stimulants, gastric proton pump inhibitors, gastric secretion inhibitors, gastroprokinetics, glucocorticoids, alpha-glucosidase inhibitors, gonad-stimulating principles, growth hormone antagonists, growth hormone inhibitors, growth hormone releasing factors, growth stimulants, hematinics, hematopoietics, hemostatics, hepatoprotectants, histamine H1-receptor antagonists, human immunodeficiency virus fusion inhibitors, or substantially any combination thereof.

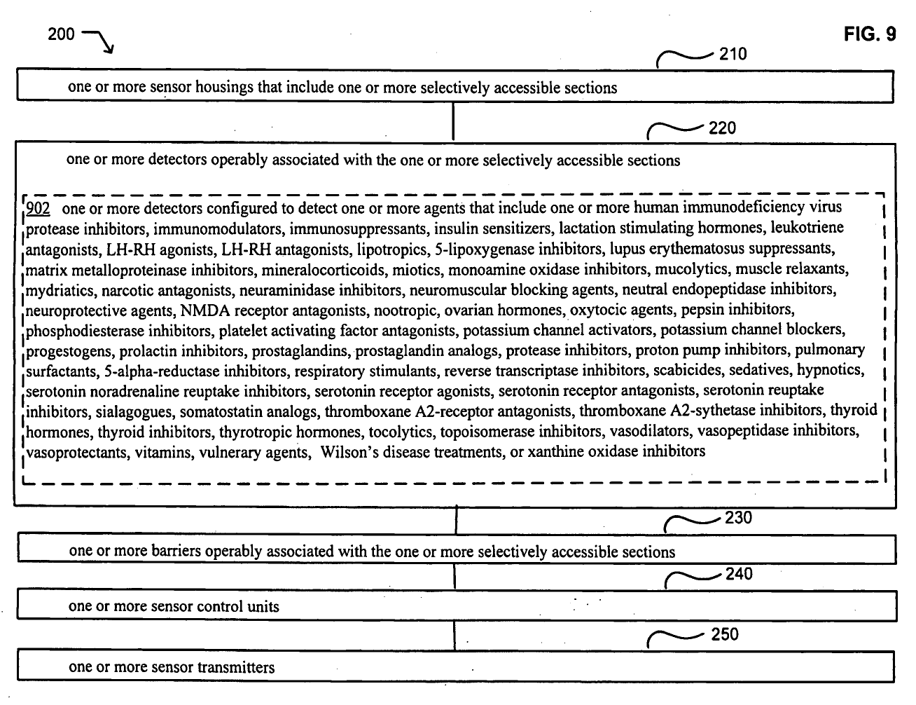

FIG. 9 illustrates alternative embodiments of embodiment 200 of sensor 102 within system 100 of FIG. 2. FIG. 9 illustrates example embodiments of module 220. Additional embodiments may include an embodiment 902.

At embodiment 902, module 220 may include one or more detectors configured to detect one or more agents that include one or more human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, or xanthine oxidase inhibitors. In some embodiments, one or more detectors 114 operably associated with the one or more selectively accessible sections 112 may include one or more detectors 114 configured to detect one or more agents that include one or more human immunodeficiency virus protease inhibitors, immunomodulators, immunosuppressants, insulin sensitizers, lactation stimulating hormones, leukotriene antagonists, LH-RH agonists, LH-RH antagonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuraminidase inhibitors, neuromuscular blocking agents, neutral endopeptidase inhibitors, neuroprotective agents, NMDA receptor antagonists, nootropic, ovarian hormones, oxytocic agents, pepsin inhibitors, phosphodiesterase inhibitors, platelet activating factor antagonists, potassium channel activators, potassium channel blockers, progestogens, prolactin inhibitors, prostaglandins, prostaglandin analogs, protease inhibitors, proton pump inhibitors, pulmonary surfactants, 5-alpha-reductase inhibitors, respiratory stimulants, reverse transcriptase inhibitors, scabicides, sedatives, hypnotics, serotonin noradrenaline reuptake inhibitors, serotonin receptor agonists, serotonin receptor antagonists, serotonin reuptake inhibitors, sialagogues, somatostatin analogs, thromboxane A2-receptor antagonists, thromboxane A2-sythetase inhibitors, thyroid hormones, thyroid inhibitors, thyrotropic hormones, tocolytics, topoisomerase inhibitors, vasodilators, vasopeptidase inhibitors, vasoprotectants, vitamins, vulnerary agents, Wilson's disease treatments, xanthine oxidase inhibitors, or substantially any combination thereof.

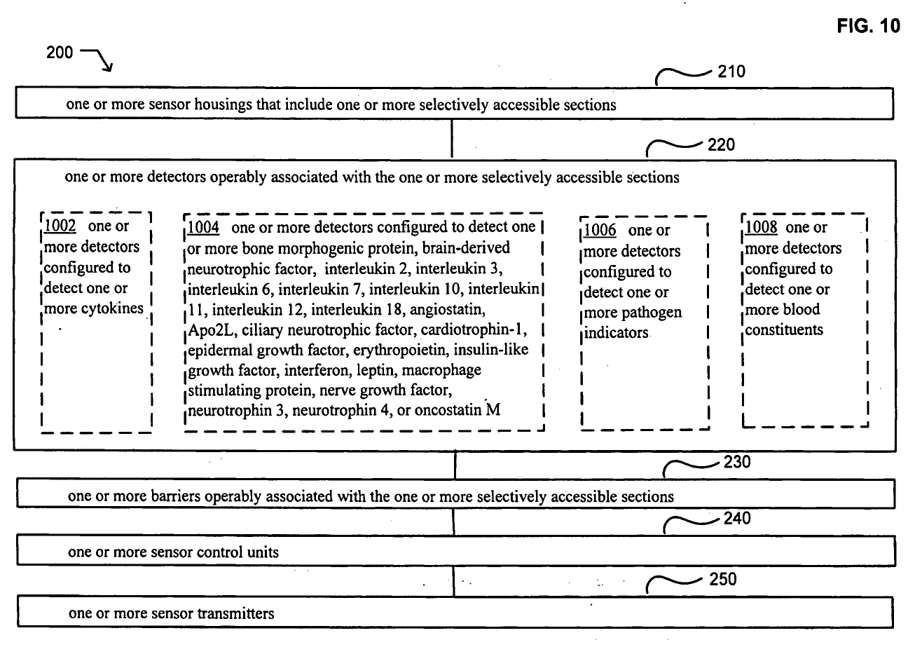

FIG. 10 illustrates alternative embodiments of embodiment 200 of sensor 102 within system 100 of FIG. 2. FIG. 10 illustrates example embodiments of module 220. Additional embodiments may include an embodiment 1002, embodiment 1004, embodiment 1006, and/or an embodiment 1008.

At embodiment 1002, module 220 may include one or more detectors configured to detect one or more cytokines. In some embodiments, one or more detectors 114 operably associated with the one or more selectively accessible sections 112 may include one or more detectors 114 configured to detect one or more cytokines. A sensor 102 may include one or more detectors 114 that are configured in numerous ways to facilitate detection of one or more cytokines. Examples of such configurations include, but are not limited to, osmotic detectors 114, electrochemical detectors 114, surface plasmon resonance detectors 114, electrochemical detectors 114, cantilever detectors 114, micro-electro-mechanical system detectors 114, and the like.

At embodiment 1004, module 220 may include one or more detectors configured to detect one or more bone morphogenic protein, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factor, interferon, leptin, macrophage stimulating protein, nerve growth factor, neurotrophin 3, neurotrophin 4, or oncostatin M. In some embodiments, one or more detectors 114 operably associated with the one or more selectively accessible sections 112 may include one or more detectors 114 configured to detect one or more bone morphogenic protein, brain-derived neurotrophic factor, interleukin 2, interleukin 3, interleukin 6, interleukin 7, interleukin 10, interleukin 11, interleukin 12, interleukin 18, angiostatin, Apo2L, ciliary neurotrophic factor, cardiotrophin-1, epidermal growth factor, erythropoietin, insulin-like growth factor, interferon, leptin, macrophage stimulating protein, nerve growth factor, neurotrophin 3, neurotrophin 4, oncostatin M, or substantially any combination thereof.

At embodiment 1006, module 220 may include one or more detectors configured to detect one or more pathogen indicators. In some embodiments, one or more detectors 114 operably associated with the one or more selectively accessible sections 112 may include one or more detectors 114 configured to detect one or more pathogen indicators. A sensor 102 may include one or more detectors 114 that are configured in numerous ways to facilitate detection of one or more pathogen indicators. Examples of such configurations include, but are not limited to, osmotic detectors 114, electrochemical detectors 114, surface plasmon resonance detectors 114, electrochemical detectors 114, cantilever detectors 114, micro-electro-mechanical system detectors 114, and the like. Numerous pathogen indicators may be detected. In some embodiments, pathogen indicators include pathogens and components of pathogens. For example, in some embodiments, pathogen indicators may include polynucleotides and/or polypeptides that are associated with a pathogen. In some embodiments, pathogen indicators may include one or more products of a pathogen. In some embodiments, pathogen indicators may include products and/or substrates that are associated with the activity of one or more pathogen associated enzymes. Examples of pathogen indicators include, but are not limited to, pathogen indicators associated with: plant pathogens, animal pathogens, human pathogens, fish pathogens, bird pathogens, and the like. Examples of such pathogens include, but are not limited to, viruses, bacteria, prions, protozoans, single-celled organisms, algae, eggs of pathogenic organisms, microbes, cysts, molds, fungus, worms, amoeba, pathogenic proteins, or substantially any combination thereof. Numerous pathogens are known and have been described (e.g., Foodborne Pathogens: Microbiology and Molecular Biology, Caister Academic Press, eds. Fratamico, Bhunia, and Smith (2005); Maizels et al., Parasite Antigens Parasite Genes: A Laboratory Manual for Molecular Parasitology, Cambridge University Press (1991); National Library of Medicine).

At embodiment 1008, module 220 may include one or more detectors configured to detect one or more blood constituents. In some embodiments, one or more detectors 114 operably associated with the one or more selectively accessible sections 112 may include one or more detectors 114 configured to detect one or more blood constituents. Examples of blood constituents include, but are not limited to, immune related constituents (antibodies, T-cells, B-cells, mast cells, helper T-cells, cytotoxic T-cells, monocytes, lymphocytes, etc.), transformed cells (e.g., cancer cells, metastatic cancer cells), cholesterol, nucleic acid (e.g., oligonucleotides, polynucleotides), peptides (e.g., polypeptides, proteins, enzymes, etc.), lipids, and the like. Detectors may be configured in numerous ways to facilitate detection of one or more blood constituents. For example, in some embodiments, one or more detectors may include one or more blood constituent binders. Examples of such blood constituent binders include, but are not limited to, antibodies (e.g., IgG, IgM, IgA, IgE, IgD), antibody fragments (e.g., Fab fragment), recombinant antibodies (e.g., humanized antibodies, single-chain antibodies, etc.), aptamers (e.g., oligonucleotides, peptides, nucleoproteins), and the like. In some embodiments, one or more detectors may include combinations of blood constituent binders.

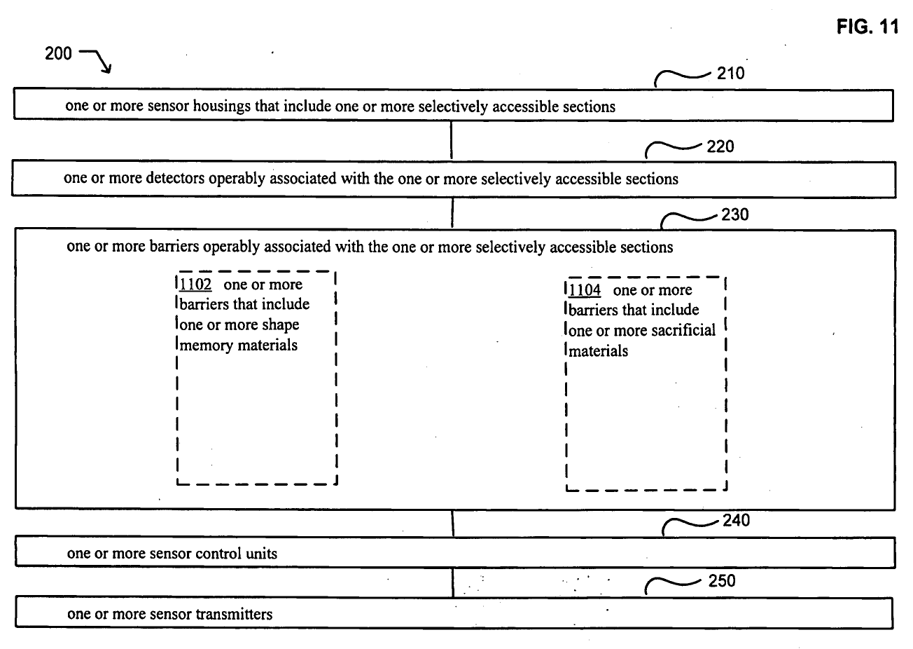

FIG. 11 illustrates alternative embodiments of embodiment 200 of sensor 102 within system 100 of FIG. 2. FIG. 11 illustrates example embodiments of module 230. Additional embodiments may include an embodiment 1102, and/or an embodiment 1104.

At embodiment 1102, module 230 may include one or more barriers that include one or more shape memory materials. In some embodiments, one or more barriers 186 operably associated with the one or more selectively accessible sections 112 may include one or more barriers 186 that include one or more shape memory materials. In some embodiments, one or more shape memory materials may be magnetic shape-memory materials. Magnetic shape-memory materials change shape in response to a magnetic field. Examples of magnetic shape-memory materials include, but are not limited to, nickel-manganese-gallium alloys, nickel-titanium alloys, copper-zinc-nickel alloys, and copper-aluminum-nickel alloys. In some embodiments, shape memory materials may be shape memory polymers. In some embodiments, shape memory polymers change shape in response to temperature. In some embodiments, a shape memory polymer may include include oligo(ε-caprolactone)diol and crystallisable oligo(ρ-dioxanone)diol. In some embodiments, a shape memory polymer may include combinations of N,N,N′,N′-Tetrakis(2-hydroxypropyl)ethylenediamine (HPED), triethanolamine (TEA), butane diol (BD), and hexamethylene diisocynate (HDI), with the following range of compositions based on 1 moles equivalent of HDI: 0.1 to 0.5 moles HPED, 0 to 0.54 moles of TEA, and 0 to 0.40 moles of BD. In some embodiments, shape memory materials may be light-induced shape-memory polymers (Lendlein et al., Letters to Nature, Nature 434:879-882 (2005). Light-induced shape-memory polymers change shape in response to light. One or more sensors 102 may include numerous types of shape memory materials.

At embodiment 1104, module 230 may include one or more barriers that include one or more sacrificial materials. In some embodiments, one or more barriers 186 operably associated with the one or more selectively accessible sections 112 may include one or more barriers 186 that include one or more sacrificial materials. In some embodiments, one or more barriers 186 may include one or more metal foils. For example, in some embodiments, one or more barriers 186 may include a sacrifical material that includes gold foil. In some embodiments, gold sacrificial material may be removed through electrochemical dissolution with a constant DC current (e.g., 35 mA/cm²) (Pan et al., Proceedings of the 26^{th} Annual International Conference of the IEEE EMBS, San Francisco, Calif., USA, Sep. 1-5, 2004).

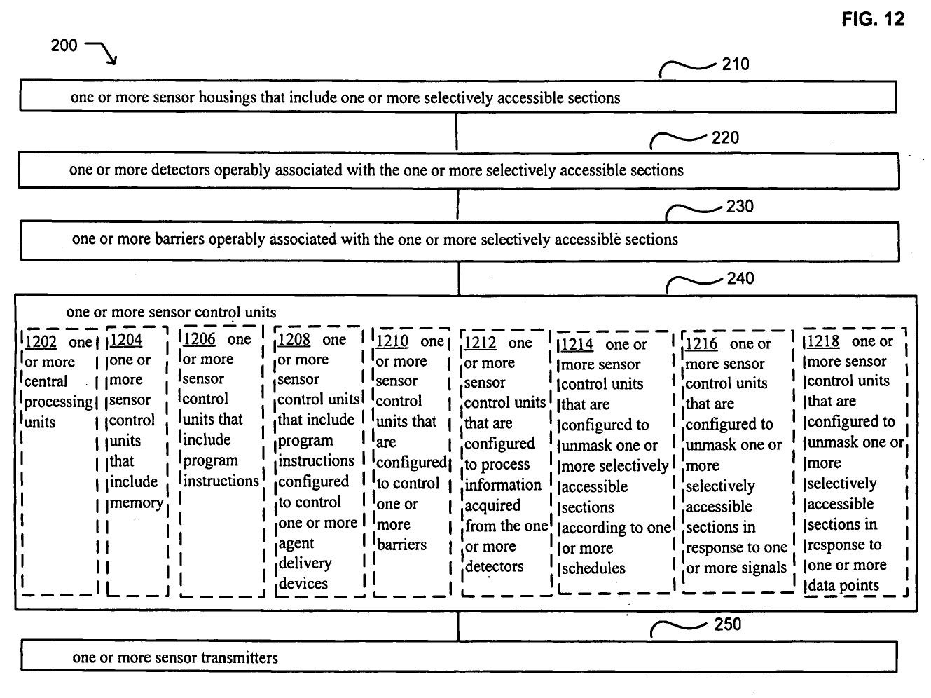

FIG. 12 illustrates alternative embodiments of embodiment 200 of sensor 102 within system 100 of FIG. 2. FIG. 12 illustrates example embodiments of module 240. Additional embodiments may include an embodiment 1202, an embodiment 1204, an embodiment 1206, an embodiment 1208, an embodiment 1210, an embodiment 1212, an embodiment 1214, an embodiment 1216, and/or an embodiment 1218.

At embodiment 1202, module 240 may include one or more central processing units. In some embodiments, one or more sensor control units 104 may include one or more central processing units. Central processing units may be configured to process numerous types of information. For example, in some embodiments, one or more central processing units may be configured to calculate one or more concentrations of one or more analytes detected by one or more sensors 102. In some embodiments, one or more central processing units may be configured to calculate one or more changes in the concentration of one or more analytes detected by one or more sensors 102. In some embodiments, one or more central processing units may be configured to calculate the amount of one or more agents 162 for administration to an individual. For example, in some embodiments, one or more central processing units may be configured to calculate the amount of agent 162 for administration to an individual that will increase the concentration of the agent 162 within the individual to reach a concentration setpoint. In some embodiments, one or more central processing units may be configured to calculate the amount of agent 162 for administration to an individual that will maintain the concentration of the agent 162 within the individual at a concentration setpoint. In some embodiments, one or more central processing units may be configured to calculate the amount of agent 162 for administration to an individual that will increase the concentration of the agent 162 within the individual to reach a concentration range. In some embodiments, one or more central processing units may be configured to calculate the amount of agent 162 for administration to an individual that will maintain the concentration of the agent 162 within the individual witin a concentration range. Accordingly, in some embodiments, a sensor 102 may include one or more central processing units that are configured in numerous ways.

At embodiment 1204, module 240 may include one or more sensor control units that include memory. In some embodiments, one or more sensor control units 104 may include one or more sensor control units 104 that include memory. One or more sensors 102 may include numerous types of memory. In some embodiments, one or more sensors 102 may include volatile memory. In some embodiments, one or more sensors 102 may include non-volatile memory. Examples of memory include, but are not limited to, read-only memory, random access memory, erasable programmable read-only memory, static random access memory, dynamic random access memory, electrically erasable programmable read-only memory, and flash memory. In some embodiments, one or more sensors 102 may include memory that is configured to include data. For example, in some embodiments, memory may be configured to include one or more look-up tables. In some embodiments, such a look-up table may include data related to one or more agents 162. For example, in some embodiments, a look-up table may include one or more amounts of one or more agents 162 for administration to an individual that are relative to the concentration of the agent 162 that is detected within the individual. Accordingly, in some embodiments, one or more sensors 102 may be configured to detect one or more agents 162 within an individual and then access one or more look-up tables to determine the amount of one or more agents 162 that may be administered to the individual to maintain the one or more agents 162 at a concentration setpoint. In some embodiments, memory may be used to store information obtained by one or more sensors 102. For example, in some embodiments, one or more sensors 102 may obtain data related to one or more concentrations of one or more agents 162 within an individual and then store the data in memory. Accordingly, memory may be used to store numerous types of information.

At embodiment 1206, module 240 may include one or more sensor control units that include program instructions. In some embodiments, one or more sensor control units 104 may include one or more sensor control units 104 that include program instructions. In some embodiments, one or more program instructions may be configured to control one or more sensors 102. For example, in some embodiments, one or more sensor control units 104 may include program instructions that are configured to regulate unmasking of selectively accessible sections 112 of one or more sensors 102 to expose one or more detectors 114. In some embodiments, program instructions may be configured to facilitate unmasking of one or more selectively accessible sections 112 of one or more sensors 102 according to one or more schedules. In some embodiments, program instructions may be configured to facilitate unmasking of one or more selectively accessible sections 112 of one or more sensors 102 at one or more times. In some embodiments, program instructions may be configured to facilitate unmasking of one or more selectively accessible sections 112 of one or more sensors 102 in response to receipt of one or more signals. For example, in some embodiments, one or more sensors 102 may receive one or more signals from one or more agent delivery devices 128 that instruct the one or more sensors 102 to detect one or more analytes. Accordingly, in some embodiments, one or more sensor control units 104 may regulate which detectors 114 are available for detection of one or more analytes and when the one or more detectors 114 are made available.

At embodiment 1208, module 240 may include one or more sensor control units that include program instructions configured to control one or more agent delivery devices. In some embodiments, one or more sensor control units 104 may include one or more sensor control units 104 that include program instructions configured to control one or more agent delivery devices 128. In some embodiments, one or more program instructions may be configured to facilitate the operation of one or more agent delivery devices 128. For example, in some embodiments, program instructions may include instructions for one or more sensors 102 to transmit one or more instructions to one or more agent delivery devices 128. In some embodiments, such instructions may include instructions to administer one or more amounts of one or more agents 162 to an individual. In some embodiments, such instructions may include instructions to stop administering one or more amounts of one or more agents 162 to an individual. In some embodiments, such instructions may include instructions to administer one or more agents 162 to an individual at one or more times. Accordingly, program instructions may include numerous types of information.

At embodiment 1210 module 240 may include one or more sensor control units that are configured to control one or more barriers. In some embodiments, one or more sensor control units 104 may include one or more sensor control units 104 that are configured to control one or more barriers 186. In some embodiments, one or more sensor control units 104 may be configured to selectively unmask one or more selectively accessible sections 112 associated with one or more sensors 102 through elimination of one or more barriers 186. For example, in some embodiments, one or more sensor control units 104 may apply an electric current to one or more barriers 186 made from gold foil that mask one or more selectively accessible sections 112 of one or more sensors 102 to cause decomposition of the one or more gold foil barriers 186 and unmask the one or more selectively accessible sections 112. In some embodiments, one or more sensor control units 104 may apply an electric current to one or more barriers 186 that include a shape memory material that mask one or more selectively accessible sections 112 of one or more sensors 102 to cause a change in conformation of the one or more barriers 186 and unmask the one or more selectively accessible sections 112. In some embodiments, one or more sensor control units 104 may apply an electric current to one or more electromagnetic barriers 186 that mask one or more selectively accessible sections 112 of one or more sensors 102 to cause the electromagnetic barrier 186 to assume an open conformation and unmask the one or more selectively accessible sections 112.

At embodiment 1212, module 240 may include one or more sensor control units that are configured to process information acquired from the one or more detectors. In some embodiments, one or more sensor control units 104 may include one or more sensor control units 104 that are configured to process information acquired from the one or more detectors 114. In some embodiments, one or more sensor control units 104 may be configured to determine one or more concentrations of one or more analytes detected by one or more detectors 114. In some embodiments, one or more sensor control units 104 may be configured to determine the amount of one or more agents 162 for administration to an individual that will increase the concentration of the one or more agents 162 within the individual to one or more levels. In some embodiments, one or more sensor control units 104 may be configured to act in response to information acquired from one or more sensors 102. For example, in some embodiments, one or more sensor control units 104 may transmit one or more internal signals 160 in response to detection of one or more analytes. In some embodiments, one or more sensor control units 104 may transmit one or more internal signals 160 in response to detection of one or more physiological conditions. Examples of physiological conditions include, but are not limited to, a heart attach, a drop in blood pressure, an increase in blood pressure, detection of one or more pathogen indicators, and the like. For example, in some embodiments, one or more sensor control units 104 may transmit one or more internal signals 160 in response to detection of a heart attack.

At embodiment 1214, module 240 may include one or more sensor control units that are configured to unmask one or more selectively accessible sections according to one or more schedules. In some embodiments, one or more sensor control units 104 may include one or more sensor control units 104 that are configured to unmask one or more selectively accessible sections 112 in response to one or more schedules. In some embodiments, one or more sensor control units 104 may be configured to unmask one or more selectively accessible sections 112 in response to one or more time schedules. In some embodiments, one or more sensor control units 104 may be configured to unmask one or more selectively accessible sections 112 in response to one or more agent administration schedules. In some embodiments, one or more sensor control units 104 may be configured to unmask one or more selectively accessible sections 112 in response to one or more time schedules in response to detection of one or more analytes. In some embodiments, one or more sensor control units 104 may be configured to unmask one or more selectively accessible sections 112 in response to one or more time schedules in response to detection of one or more physiological conditions.

At embodiment 1216, module 240 may include one or more sensor control units that are configured to unmask one or more selectively accessible sections in response to one or more signals. In some embodiments, one or more sensor control units 104 may include one or more sensor control units 104 that are configured to unmask one or more selectively accessible sections 112 in response to one or more internal signals 160. In some embodiments, one or more sensor control units 104 may be configured to unmask one or more selectively accessible sections 112 in response to one or more internal signals 160 received from one or more agent delivery devices 146. Accordingly, in some embodiments, one or more sensor control units 104 may be configured to operate within a feedback loop with one or more agent delivery units 146.

At embodiment 1218, module 240 may include one or more sensor control units that are configured to unmask one or more selectively accessible sections in response to one or more data points. In some embodiments, one or more sensor control units 104 may include one or more sensor control units 104 that are configured to unmask one or more selectively accessible sections 112 in response to one or more data points. For example, in some embodiments, one or more sensor control units 104 may unmask one or more selectively accessible sections 112 that include one or more nitric oxide detectors 114 in response to data indicating a drop in blood pressure. Accordingly, one or more sensor control units 104 may respond to numerous types of data points.

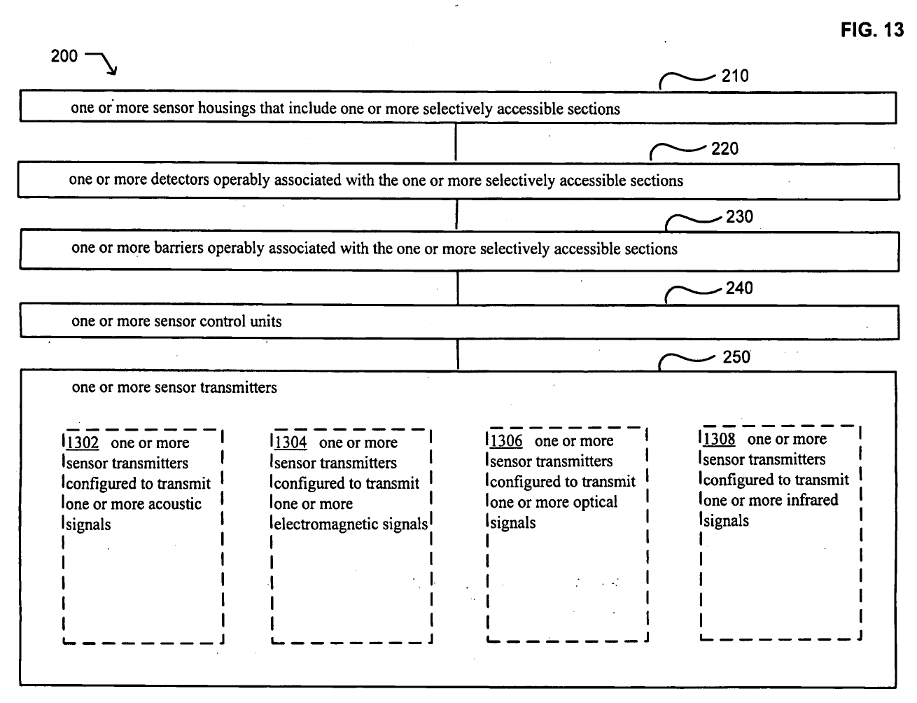

FIG. 13 illustrates alternative embodiments of embodiment 200 of sensor 102 within system 100 of FIG. 2. FIG. 13 illustrates example embodiments of module 250. Additional embodiments may include an embodiment 1302, an embodiment 1304, an embodiment 1306, and/or an embodiment 1308.

At embodiment 1302, module 250 may include one or more sensor transmitters configured to transmit one or more acoustic signals. In some embodiments, one or more sensor transmitters 126 may include one or more sensor transmitters 126 configured to transmit one or more acoustic signals. Numerous types of acoustic transmitters and receivers may be used to send and receive signals. Methods to fabricate acoustic transmitters and receivers are known and have been described (e.g., U.S. Pat. Nos. 7,301,473; 4,142,478; 3,978,940; 4,002,897; and 6,488,116).

At embodiment 1304, module 250 may include one or more sensor transmitters configured to transmit one or more electromagnetic signals. In some embodiments, one or more sensor transmitters 126 may include one or more sensor transmitters 126 configured to transmit one or more electromagnetic signals. In some embodiments, one or more electromagnetic signals may be received through use of an optical fiber (e.g., U.S. Pat. No. 5,307,195). In some embodiments, one or more electromagnetic signals may be received through use of a conductive wire (e.g., U.S. Pat. No. 5,122,773). Devices that are configured to receive one or more electromagnetic signals have been described (e.g., U.S. Pat. No. 6,993,259).

At embodiment 1306, module 250 may include one or more sensor transmitters configured to transmit one or more optical signals. In some embodiments, one or more sensor transmitters 126 may include one or more sensor transmitters 126 configured to transmit one or more optical signals. Numerous types of optical transmitters and receivers may be used to send and receive signals. Methods to fabricate optical transmitters and receivers are known and have been described (e.g., U.S. Pat. Nos. 5,170,274; 5,949,566; 6,192,060; 5,307,196; and 6,304,357).

At embodiment 1308, module 250 may include one or more sensor transmitters configured to transmit one or more infrared signals. In some embodiments, one or more sensor transmitters 126 may include one or more sensor transmitters 126 configured to transmit one or more infrared signals. Numerous types of infrared transmitters and receivers may be used to send and receive signals. Methods to fabricate infrared transmitters and receivers are known and have been described (e.g., U.S. Pat. Nos. 4,371,814; 5,359,448; and 5,331,450).

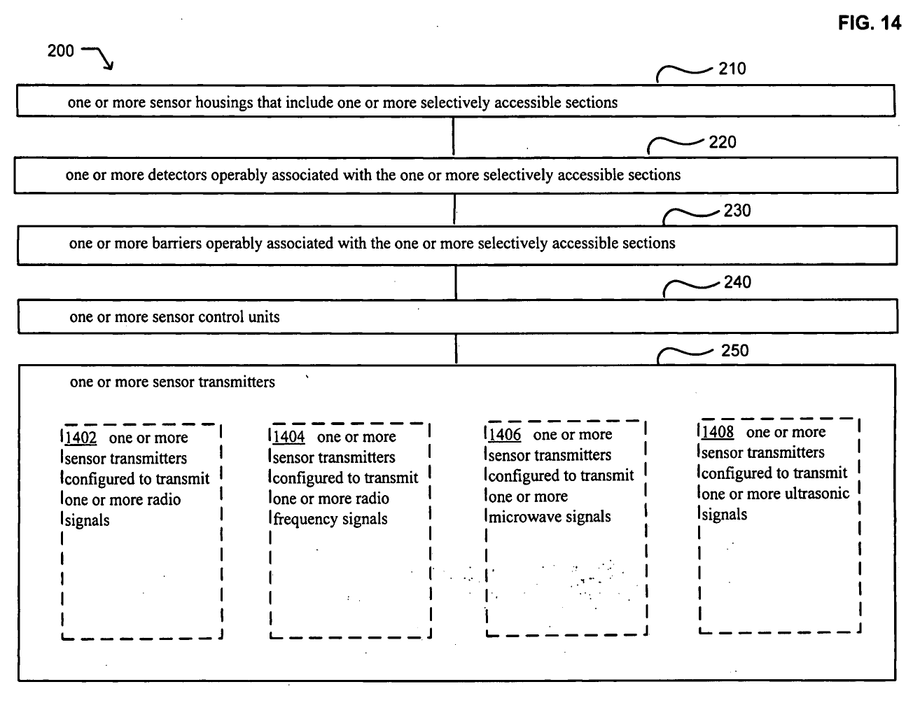

FIG. 14 illustrates alternative embodiments of embodiment 200 of sensor 102 within system 100 of FIG. 2. FIG. 14 illustrates example embodiments of module 250. Additional embodiments may include an embodiment 1402, an embodiment 1404, an embodiment 1406, and/or an embodiment 1408.

At embodiment 1402, module 250 may include one or more sensor transmitters configured to transmit one or more radio signals. In some embodiments, one or more sensor transmitters 126 may include one or more sensor transmitters 126 configured to transmit one or more radio signals. Numerous types of radio transmitters and receivers may be used to send and receive signals. Methods to fabricate radio transmitters and receivers are known and have been described (e.g., U.S. Pat. Nos. 5,826,177; 4,355,401; 5,241,561; and 5,353,311).

At embodiment 1404, module 250 may include one or more sensor transmitters configured to transmit one or more radio frequency signals. In some embodiments, one or more sensor transmitters 126 may include one or more sensor transmitters 126 configured to transmit one or more radio frequency signals. Methods to fabricate devices that transmit and receive radio frequency signals are known and have been described (e.g., U.S. Pat. Nos. 7,171,175; 7,031,676; 6,587,511; 4,258,436; 4,047,121; 4,013,966).

At embodiment 1406, module 250 may include one or more sensor transmitters configured to transmit one or more microwave signals. In some embodiments, one or more sensor transmitters 126 may include one or more sensor transmitters 126 configured to transmit one or more microwave signals. Devices that are configured to transmit and receive one or more microwave signals have been described (e.g., U.S. Pat. Nos. 4,196,393; 4,032,859; 4,121,163; 5,053,722).

At embodiment 1408, module 250 may include one or more sensor transmitters configured to transmit one or more ultrasonic signals. In some embodiments, one or more sensor transmitters 126 may include one or more sensor transmitters 126 configured to transmit one or more ultrasonic signals. Numerous types of ultrasonic transmitters and receivers may be used to send and receive signals. Methods to fabricate ultrasonic transmitters and receivers are known and have been described (e.g., U.S. Pat. Nos. 7,162,930; 6,854,338; 6,087,760; 6,212,936; 4,326,274; and 5,483,226).

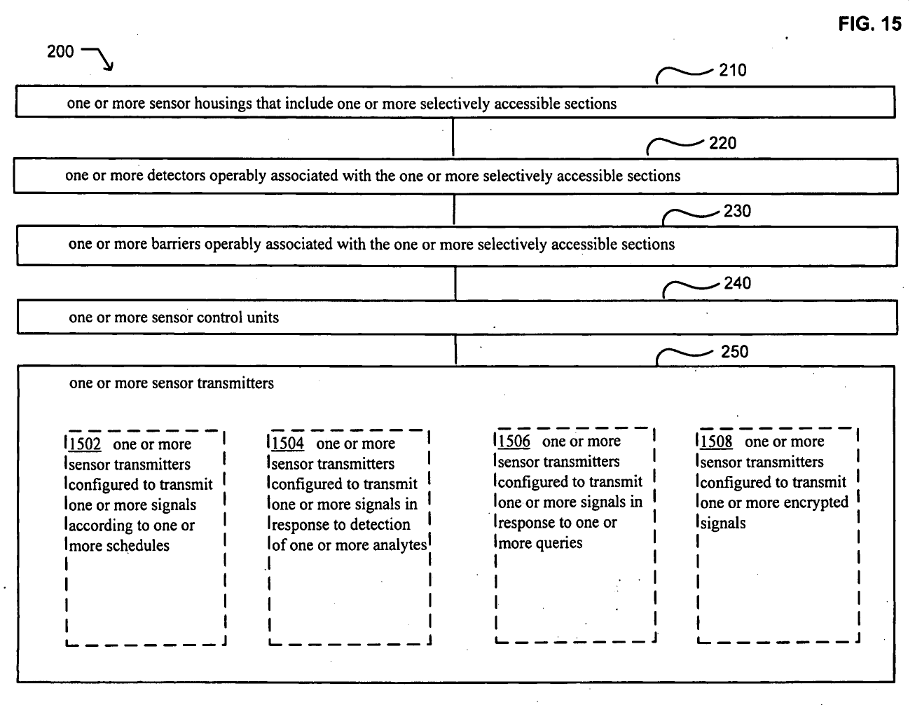

FIG. 15 illustrates alternative embodiments of embodiment 200 of sensor 102 within system 100 of FIG. 2. FIG. 15 illustrates example embodiments of module 250. Additional embodiments may include an embodiment 1502, an embodiment 1504, an embodiment 1506, and/or an embodiment 1508.

At embodiment 1502, module 250 may include one or more sensor transmitters configured to transmit one or more signals according to one or more schedules. In some embodiments, one or more sensor transmitters 126 may include one or more sensor transmitters 126 configured to transmit one or more signals according to one or more schedules. In some embodiments, one or more sensor transmitters 126 may be configured to transmit one or more signals according to one or more time schedules. In some embodiments, one or more sensor transmitters 126 may be configured to transmit one or more signals according to one or more agent administration schedules.

At embodiment 1504, module 250 may include one or more sensor transmitters configured to transmit one or more signals in response to detection of one or more analytes. In some embodiments, one or more sensor transmitters 126 may include one or more sensor transmitters 126 configured to transmit one or more signals in response to detection of one or more analytes. In some embodiments, one or more sensor transmitters 126 may be configured to transmit one or more signals in response to detecting the presence of one or more analytes. In some embodiments, one or more sensor transmitters 126 may be configured to transmit one or more signals in response to detecting an absence of one or more analytes. In some embodiments, one or more sensor transmitters 126 may be configured to transmit one or more signals in response to detecting one or more concentrations of one or more analytes. For example, in some embodiments, one or more sensor transmitters 126 may transmit one or more messages in response to detection of one or more analytes at one or more concentrations that are above a threshold value. In some embodiments, one or more sensor transmitters 126 may transmit one or more messages in response to detection of one or more analytes at one or more concentrations that are below a threshold value.

At embodiment 1506, module 250 may include one or more sensor transmitters configured to transmit one or more signals in response to one or more queries. In some embodiments, one or more sensor transmitters 126 may include one or more sensor transmitters 126 configured to transmit one or more signals in response to one or more queries. In some embodiments, a sensor 102 may be configured to receive one or more signals that include one or more queries and then transmit one or more signals in response to the one or more queries. For example, in some embodiments, one or more sensors 102 may receive one or more queries from one or more agent delivery devices 128 and transmit one or more signals in response to the one or more queries. Accordingly, in some embodiments, one or more sensors 102 may be in communication with one or more agent delivery devices 128 to form a feedback loop.

At embodiment 1508, module 250 may include one or more sensor transmitters configured to transmit one or more encrypted signals. In some embodiments, one or more sensor transmitters 126 may include one or more sensor transmitters 126 configured to transmit one or more encrypted signals. One or more sensor transmitter 126 may utilize numerous types of encryption.

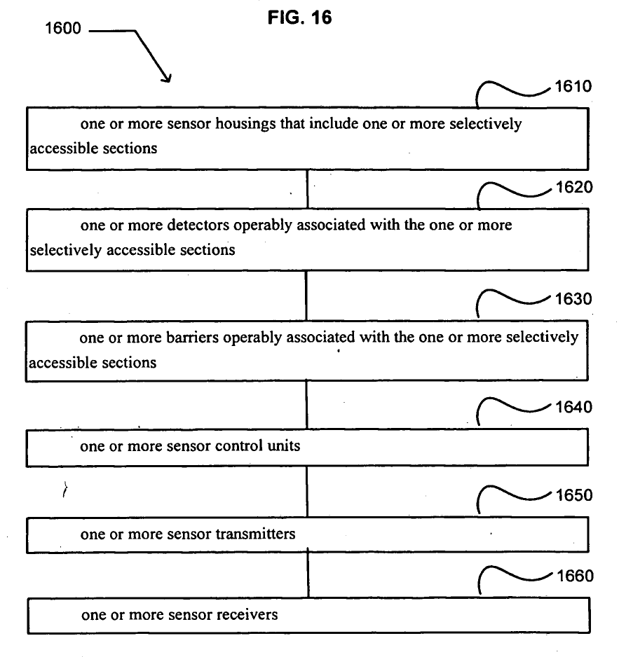

FIG. 16 illustrates embodiment 1600 of sensor 102 within system 100. In FIG. 16, discussion and explanation may be provided with respect to the above-described example of FIG. 1, and/or with respect to other examples and contexts. In some embodiments, modules 210, 220, 230, 240 and 250 of FIG. 2 may correspond to modules 1610, 1620, 1630, 1640 and 1650 of FIG. 16. However, it should be understood that the modules may execute operations in a number of other environments and contexts, and/or modified versions of FIG. 1. Also, although the various modules are presented in the sequence(s) illustrated, it should be understood that the various modules may be configured in numerous orientations.

The embodiment 1600 may include module 1610 that includes one or more sensor housings that include one or more selectively accessible sections. In some embodiments, one or more sensors 102 may include one or more sensor housings 184 that include one or more selectively accessible sections. Numerous types of material may be used to fabricate one or more sensor housings 184. Examples of such materials include, but are not limited to, metals, metal alloys, plastics, ceramics, polymeric materials, and the like. In some embodiments, one or more sensor housings 184 may include one or more types of materials. For example, in some embodiments, one or more sensor housings 184 may be made of metal and then coated with a polymeric coating. Accordingly, sensor housings 184 may be made of numerous materials and combinations of materials. A sensor 102 may include one or more sensor housings 184 that include one or more selectively accessible sections 112. In some embodiments, a sensor 102 may include one or more sensor housings 184 that include one or more selectively accessible sections 112 that contain one or more detectors 114 that are configured to detect the same type of analyte. In some embodiments, a sensor 102 may include one or more sensor housings 184 that include one or more selectively accessible sections 112 that contain one or more detectors 114 that are configured to detect different types of analytes. In some embodiments, a sensor 102 may include one or more sensor housings 184 that include circuitry for association of one or more sensor receivers 124 with the sensor 102. In some embodiments, a sensor 102 may include one or more sensor housings 184 that include circuitry for association of one or more sensor transmitters 126 with the sensor 102. In some embodiments, a sensor 102 may include one or more sensor housings 184 that include circuitry for association of one or more detectors 114 with the sensor 102. In some embodiments, a sensor 102 may include one or more sensor housings 184 that include circuitry for association of one or more sensor control units 104 with the sensor 102. In some embodiments, a sensor 102 may include one or more sensor housings 184 that include circuitry for association of one or more sensor electromagnetic receivers 118 with the sensor 102. In some embodiments, a sensor 102 may include one or more sensor housings 184 that include circuitry for association of one or more sensor capacitors 122 with the sensor 102. In some embodiments, a sensor 102 may include one or more sensor housings 184 that include circuitry for association of one or more sensor batteries 120 with the sensor 102. In some embodiments, a sensor 102 may include one or more sensor housings 184 that include circuitry for association of one or more processors with the sensor 102. In some embodiments, a sensor 102 may include one or more sensor housings 184 that include circuitry for association of one or more sensor receivers 124, sensor transmitters 126, sensor electromagnetic receivers 118, sensor capacitors 122, sensor control units 104, processors, analyte detection memories 110, detectors 114, or substantially any combination thereof with the sensor 102. In some embodiments, a sensor 102 may include one or more sensor housings 184 that include selectively accessible sections 112 that are associated with one or more barriers 186. Numerous types of barriers 186 may be associated with the one or more selectively accessible sections 112. Examples of such barriers 186 include, but are not limited to, metals, shape memory polymers, mechanical doors, electromagnetic valves, and combinations thereof. In some embodiments, one or more sensor housings 184 may include the same type of barrier 186. In some embodiments, one or more sensor housings 184 may include different types of barriers 186.

The embodiment 1600 may include module 1620 that includes one or more detectors operably associated with the one or more selectively accessible sections. In some embodiments, one or more sensors 102 may include one or more detectors 114 operably associated with the one or more selectively accessible sections 112. Numerous types of detectors 114 may be associated with a sensor 102. Examples of such detectors 114 include, but are not limited to, osmotic detectors 114, micro-electro-mechanical system detectors 114, cantilever detectors 114, electrochemical detectors 114, surface plasmon resonance detectors 114, electrodes, detectors 114 configured to detect nitric oxide, detectors 114 configured to detect glucose, detectors 114 configured to detect one or more hormones, detectors 114 configured to detect one or more pharmaceutical agents 162, detectors 114 configured to detect one or more cytokines, detectors 114 configured to detect one or more pathogen indicators, or substantially any combination thereof. In some embodiments, a sensor 102 may include one type of detector 114. In some embodiments, a sensor 102 may include one or more types of detectors 114.

The embodiment 1600 may include module 1630 that includes one or more barriers operably associated with the one or more selectively accessible sections. In some embodiments, one or more sensors 102 may include one or more barriers 186 operably associated with the one or more selectively accessible sections 112. In some embodiments, one or more selectively accessible sections 112 may be operably associated with one or more barriers 186 that sequester the selectively accessible section 112 from an outside environment. For example, in some embodiments, a selectively accessible section 112 may be sequestered from the outside environment by an impermeable metal foil barrier 186. In some embodiments, one or more selectively accessible sections 112 may be operably associated with one or more barriers 186 that selectively sequester the selectively accessible section 112 from an outside environment. For example, in some embodiments, a selectively accessible section 112 may be selectively sequestered from the outside environment by a selectively permeable metal foil barrier 186 (e.g., a perforated gold foil that is selectively permeable based on molecular weight).

The embodiment 1600 may include module 1640 that includes one or more sensor control units. In some embodiments, one or more sensors 102 may include one or more sensor control units 104. In some embodiments, a sensor 102 may include one or more sensor control units 104 that include one or more analyte detection processors 106. In some embodiments, a sensor 102 may include one or more sensor control units 104 that include one or more analyte detection memories 110. In some embodiments, a sensor 102 may include one or more sensor control units 104 that include analyte detection logic 108. In some embodiments, a sensor 102 may include one or more sensor control units 104 that are configured to facilitate unmasking of one or more selectively accessible sections 112 associated with the sensor 102. In some embodiments, a sensor 102 may include one or more sensor control units 104 that are configured to control the operation of one or more sensor transmitters 126. In some embodiments, a sensor 102 may include one or more sensor control units 104 that are configured to control the operation of one or more sensor receivers 124. In some embodiments, a sensor 102 may include one or more sensor control units 104 that are configured to control the operation of one or more sensor power sources 116. In some embodiments, a sensor 102 may include one or more sensor control units 104 that are configured to control the operation of one or more sensor detectors 114.

The embodiment 1600 may include module 1650 that includes one or more sensor transmitters. In some embodiments, one or more sensors 102 may include one or more sensor transmitters 126. A sensor 102 may include one or more sensor transmitters 126 that are configured to transmit numerous types of signals. Examples of such signals include, but are not limited to, acoustic signals, electromagnetic signals, optical signals, infrared signals, radio signals, radio frequency signals, microwave signals, ultrasonic signals, or substantially any combination thereof. In some embodiments, one or more sensor transmitters 126 may be configured to transmit one or more signals according to one or more schedules. In some embodiments, one or more schedules may be time schedules. In some embodiments, one or more schedules may be agent administration schedules. In some embodiments, one or more sensor transmitters 126 may be configured to transmit one or more signals in response to detection of one or more analytes. For example, in some embodiments, one or more sensor transmitters 126 may transmit one or more signals if one or more analytes are detected. In some embodiments, one or more sensor transmitters 126 may transmit one or more signals if one or more analytes are not detected. In some embodiments, one or more sensor transmitters 126 may transmit one or more signals in response to one or more queries. For example, in some embodiments, one or more sensor transmitters 126 may transmit one or more signals in response to a query from one or more agent delivery devices 128.

The embodiment 1600 may include module 1660 that includes one or more sensor receivers. In some embodiments, one or more sensors 102 may include one or more sensor receivers 124. A sensor 102 may include one or more sensor receivers 124 that are configured to receive numerous types of signals. Examples of such signals include, but are not limited to, acoustic signals, electromagnetic signals, optical signals, infrared signals, radio signals, radio frequency signals, microwave signals, ultrasonic signals, or substantially any combination thereof. In some embodiments, one or more sensor receivers 124 may be configured to receive one or more signals according to one or more schedules. In some embodiments, one or more schedules may be time schedules. In some embodiments, one or more schedules may be agent administration schedules. In some embodiments, one or more sensor receivers 124 may be configured to receive one or more signals in response to detection of one or more analytes. For example, in some embodiments, one or more sensor receivers 124 may receive one or more signals if one or more analytes are detected. In some embodiments, one or more sensor receivers 124 may receive one or more signals if one or more analytes are not detected. In some embodiments, one or more sensor receivers 124 may receive one or more signals in response to one or more queries. For example, in some embodiments, one or more sensor receivers 124 may receive one or more signals in response to a query from one or more agent delivery devices 128.

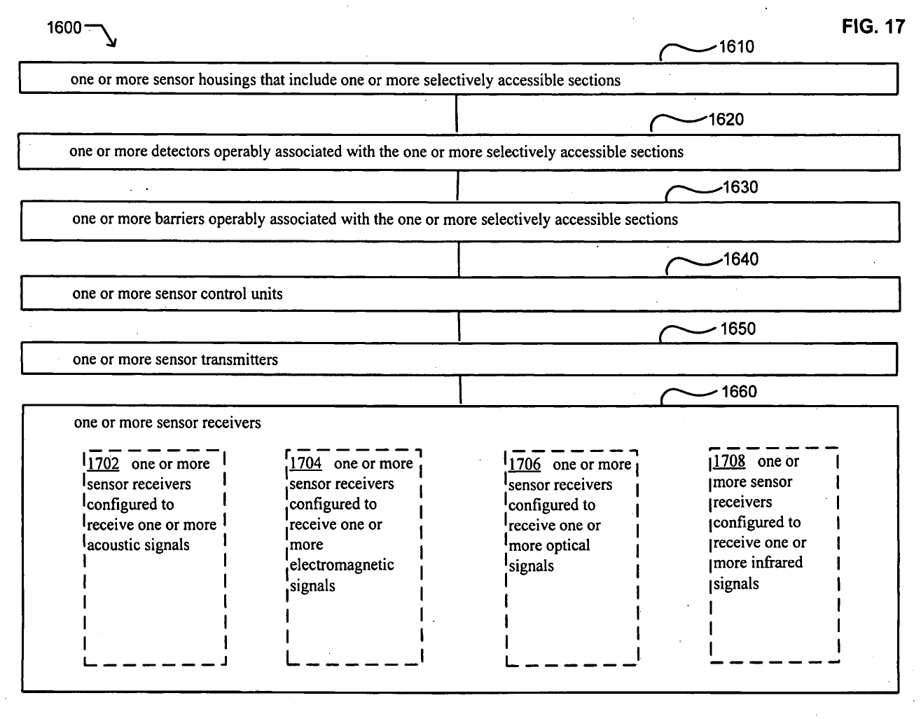

FIG. 17 illustrates alternative embodiments of embodiment 1600 of sensor 102 within system 1600 of FIG. 16. FIG. 17 illustrates example embodiments of module 1660. Additional embodiments may include an embodiment 1702, an embodiment 1704, an embodiment 1706, and/or an embodiment 1708.

At embodiment 1702, module 1660 may include one or more sensor receivers configured to receive one or more acoustic signals. In some embodiments, one or more sensor receivers 124 may include one or more sensor receivers 124 configured to receive one or more acoustic signals. Numerous types of acoustic transmitters and receivers may be used to send and receive signals. Methods to fabricate infrared transmitters and receivers are known and have been described (e.g., U.S. Pat. Nos. 7,301,473; 4,142,478; 3,978,940; 4,002,897 and 6,488,116).

At embodiment 1704, module 1660 may include one or more sensor receivers configured to receive one or more electromagnetic signals. In some embodiments, one or more sensor receivers 124 may include one or more sensor receivers 124 configured to receive one or more electromagnetic signals. In some embodiments, one or more electromagnetic signals may be received through use of an optical fiber (e.g., U.S. Pat. No. 5,307,195). In some embodiments, one or more electromagnetic signals may be received through use of a conductive wire (e.g., U.S. Pat. No. 5,122,773). Devices that are configured to receive one or more electromagnetic signals have been described (e.g., U.S. Pat. No. 6,993,259).

At embodiment 1706, module 1660 may include one or more sensor receivers configured to receive one or more optical signals. In some embodiments, one or more sensor receivers 124 may include one or more sensor receivers 124 configured to receive one or more optical signals. Numerous types of optical transmitters and receivers may be used to send and receive signals. Methods to fabricate optical transmitters and receivers are known and have been described (e.g., U.S. Pat. Nos. 5,170,274; 5,949,566; 6,192,060; 5,307,196 and 6,304,357).

At embodiment 1708, module 1660 may include one or more sensor receivers configured to receive one or more infrared signals. In some embodiments, one or more sensor receivers 124 may include one or more sensor receivers 124 configured to receive one or more infrared signals. Numerous types of infrared transmitters and receivers may be used to send and receive signals. Methods to fabricate infrared transmitters and receivers are known and have been described (e.g., U.S. Pat. Nos. 4,371,814; 5,359,448 and 5,331,450).

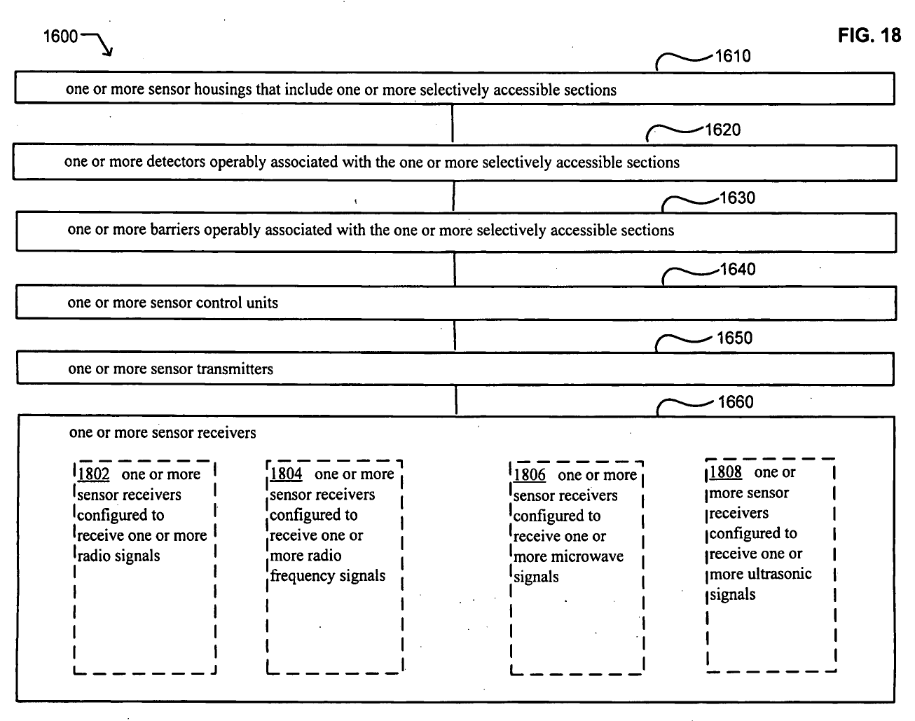

FIG. 18 illustrates alternative embodiments of embodiment 1600 of sensor 102 within system 1600 of FIG. 16. FIG. 18 illustrates example embodiments of module 1660. Additional embodiments may include an embodiment 1802, an embodiment 1804, an embodiment 1806, and/or an embodiment 1808.

At embodiment 1802, module 1660 may include one or more sensor receivers configured to receive one or more radio signals. In some embodiments, one or more sensor receivers 124 may include one or more sensor receivers 124 configured to receive one or more radio signals. Numerous types of radio transmitters and receivers may be used to send and receive signals. Methods to fabricate radio transmitters and receivers are known and have been described (e.g., U.S. Pat. Nos. 5,826,177; 4,355,401; 5,241,561 and 5,353,311).

At embodiment 1804, module 1660 may include one or more sensor receivers configured to receive one or more radio frequency signals. In some embodiments, one or more sensor receivers 124 may include one or more sensor receivers 124 configured to receive one or more radio frequency signals. Methods to fabricate devices that transmit and receive radio frequency signals are known and have been described (e.g., U.S. Pat. Nos. 7,171,175; 7,031,676; 6,587,511; 4,258,436; 4,047,121; 4,013,966; 6,535,766 and 6,868,288).

At embodiment 1806, module 1660 may include one or more sensor receivers configured to receive one or more microwave signals. In some embodiments, one or more sensor receivers 124 may include one or more sensor receivers 124 configured to receive one or more microwave signals. Devices that are configured to transmit and receive one or more microwave signals have been described (e.g., U.S. Pat. Nos. 4,196,393; 4,032,859; 4,121,163 and 5,053,722).

At embodiment 1808, module 1660 may include one or more sensor receivers configured to receive one or more ultrasonic signals. In some embodiments, one or more sensor receivers 124 may include one or more sensor receivers 124 configured to receive one or more ultrasonic signals. Numerous types of ultrasonic transmitters and receivers may be used to send and receive signals. Methods to fabricate ultrasonic transmitters and receivers are known and have been described (e.g., U.S. Pat. Nos. 7,162,930; 6,854,338; 6,087,760; 6,212,936; 4,326,274 and 5,483,226).

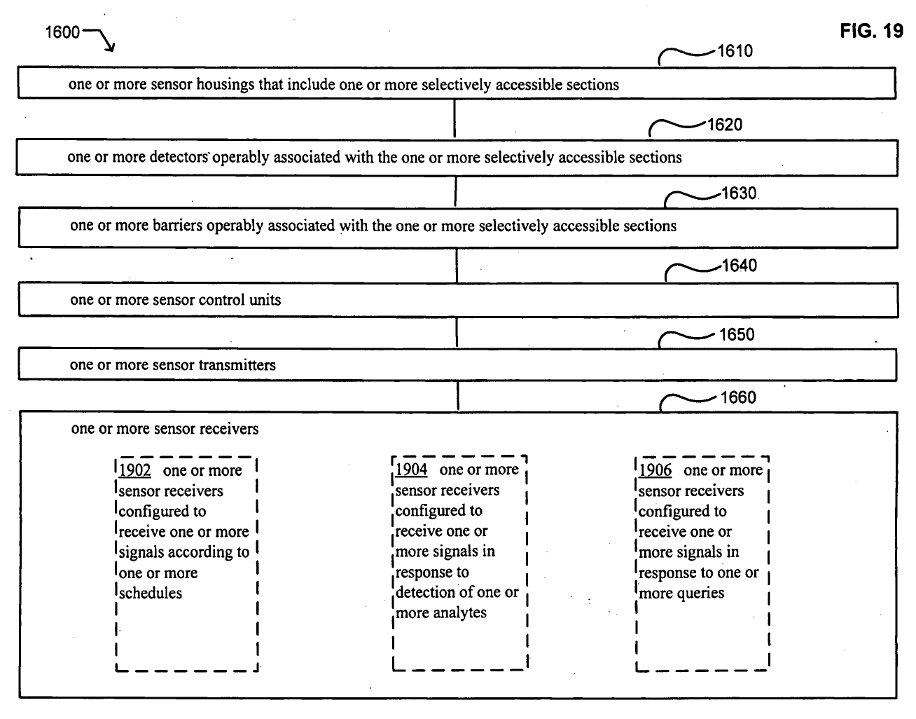

FIG. 19 illustrates alternative embodiments of embodiment 1600 of sensor 102 within system 1600 of FIG. 16. FIG. 19 illustrates example embodiments of module 1660. Additional embodiments may include an embodiment 1902, an embodiment 1904, and/or an embodiment 1906.

At embodiment 1902, module 1660 may include one or more sensor receivers configured to receive one or more signals according to one or more schedules. In some embodiments, one or more sensor receivers 124 may include one or more sensor receivers 124 configured to receive one or more signals according to one or more schedules. In some embodiments, one or more sensor receivers 124 may be configured to receive one or more signals according to one or more time schedules. In some embodiments, one or more sensor receivers 124 may be configured to receive one or more signals according to one or more agent administration schedules.

At embodiment 1904, module 1660 may include one or more sensor receivers configured to receive one or more signals in response to detection of one or more analytes. In some embodiments, one or more sensors 102 may include one or more sensor receivers 124 configured to receive one or more signals in response to detection of one or more analytes. In some embodiments, one or more sensor receivers 124 may be configured to receive one or more signals in response to the detection of one or more analytes. In some embodiments, one or more sensor receivers 124 may be configured to receive one or more signals in response to an absence of one or more analytes. In some embodiments, one or more sensor receivers 124 may be configured to receive one or more signals in response to the detection of one or more concentrations of one or more analytes. For example, in some embodiments, one or more sensor receivers 124 may receive one or more messages in response to the detection of one or more analytes at one or more concentrations that are above a threshold value. In some embodiments, one or more sensor receivers 124 may receive one or more messages in response to the detection of one or more analytes at one or more concentrations that are below a threshold value.

At embodiment 1906, module 1660 may include one or more sensor receivers configured to receive one or more signals in response to one or more queries. In some embodiments, one or more sensor receivers 124 may include one or more sensor receivers 124 configured to receive one or more signals in response to one or more queries. In some embodiments, a sensor 102 may be configured to receive one or more signals that include one or more queries and then transmit one or more signals in response to the one or more queries. For example, in some embodiments, one or more sensors 102 may receive one or more queries from one or more agent delivery devices 128 and transmit one or more signals in response to the one or more queries. Accordingly, in some embodiments, one or more sensors 102 may be in communication with one or more agent delivery devices 128 to form a feedback loop.

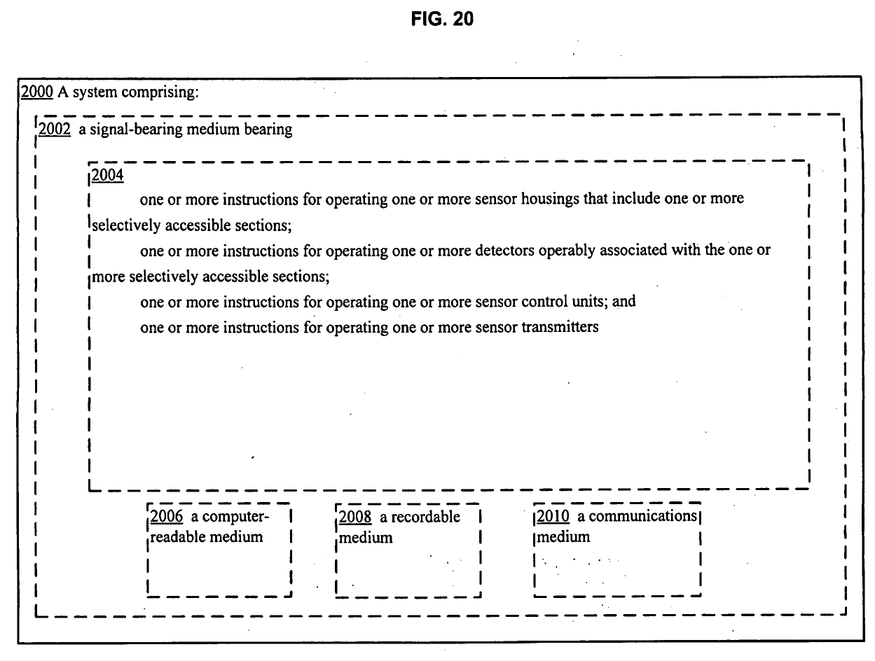

FIG. 20 illustrates a partial view of a system 2000 that includes a computer program 2004 for executing a computer process on a computing device. An embodiment of system 2000 is provided using a signal-bearing medium 2002 bearing one or more instructions for operating one or more sensor housings 184 that include one or more selectively accessible sections 112; one or more instructions for operating one or more detectors 114 operably associated with the one or more selectively accessible sections 112; one or more instructions for operating one or more sensor control units 104; and one or more instructions for operating one or more sensor transmitters 126. The one or more instructions may be, for example, computer executable and/or logic-implemented instructions. In some embodiments, the signal-bearing medium 2002 may include a computer-readable medium 2006. In some embodiments, the signal-bearing medium 2002 may include a recordable medium 2008. In some embodiments, the signal-bearing medium 2002 may include a communications medium 2010.

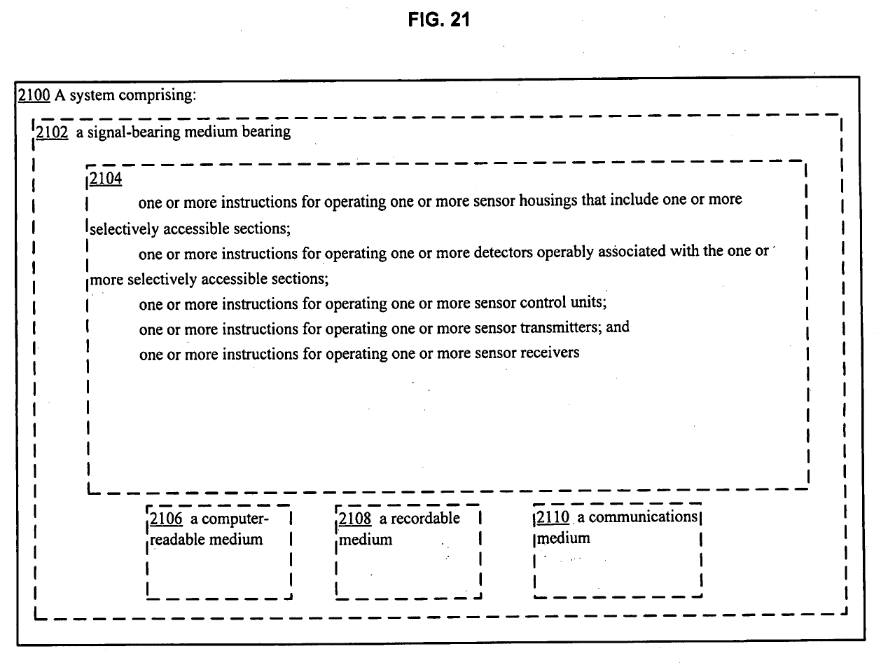

FIG. 21 illustrates a partial view of a system 2100 that includes a computer program 2104 for executing a computer process on a computing device. An embodiment of system 2100 is provided using a signal-bearing medium 2102 bearing one or more instructions for operating one or more sensor housings 184 that include one or more selectively accessible sections 112; one or more instructions for operating one or more detectors 114 operably associated with the one or more selectively accessible sections 112; one or more instructions for operating one or more sensor control units 104; one or more instructions for operating one or more sensor transmitters 126; and one or more instructions for operating one or more sensor receivers 124. The one or more instructions may be, for example, computer executable and/or logic-implemented instructions. In some embodiments, the signal-bearing medium 2102 may include a computer-readable medium 2106. In some embodiments, the signal-bearing medium 2102 may include a recordable medium 2108. In some embodiments, the signal-bearing medium 2102 may include a communications medium 2110.

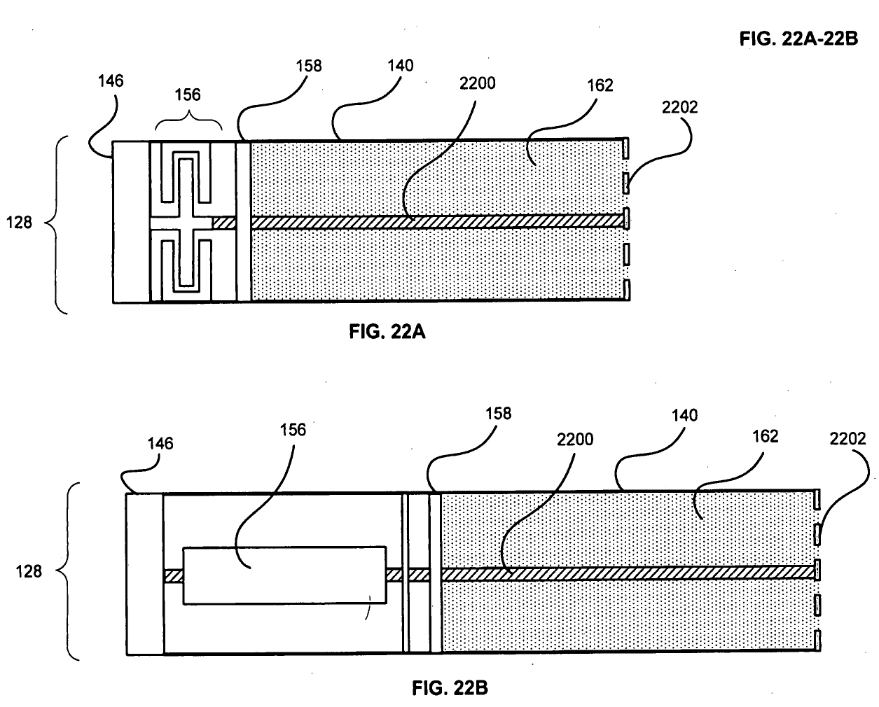

FIG. 22A illustrates an embodiment of an agent delivery device 128 that includes a stepper motor 156 that is operably coupled to a moveable member 158 through a threaded member 2200. The agent delivery device 128 includes a device housing 140, an agent delivery control unit 146, one or more agents 162, and an agent permeable barrier 2202.

FIG. 22B illustrates an embodiment of an agent delivery device 128 that includes a squiggle motor 156 that is operably coupled to a moveable member 158 through a threaded member 2200. The agent delivery device 128 includes a device housing 140, an agent delivery control unit 146, one or more agents 162, and an agent permeable barrier 2202.

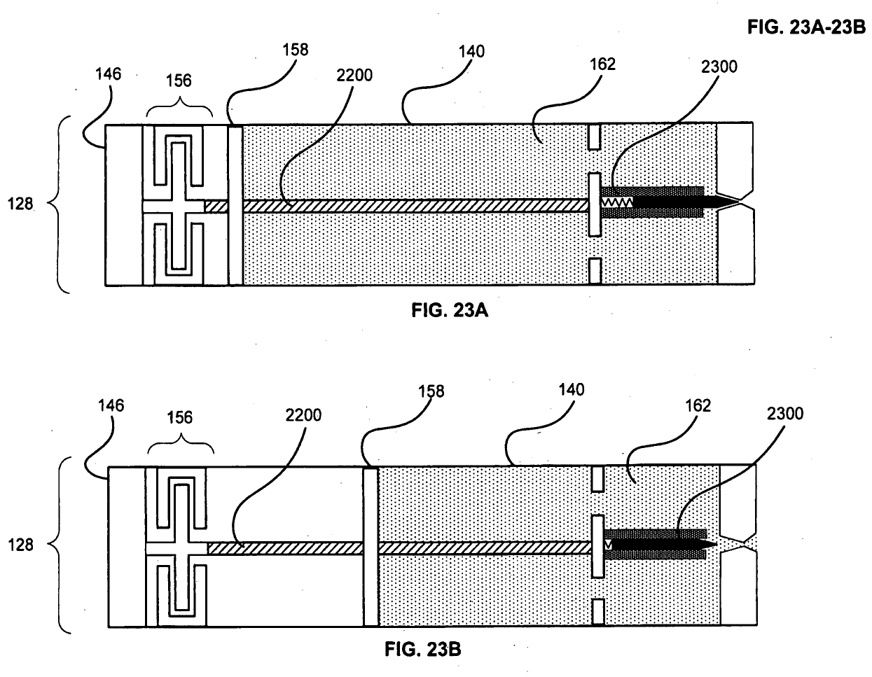

FIG. 23A illustrates an embodiment of an agent delivery device 128 that includes a stepper motor 156 that is operably coupled to a moveable member 158 through a threaded member 2200. The agent delivery device 128 includes a device housing 140, an agent delivery control unit 146, one or more agents 162, and an electromagnetic exit port 2300 that is shown in the closed position.

FIG. 23B illustrates an embodiment of an agent delivery device 128 that includes a stepper motor 156 that is operably coupled to a moveable member 158 through a threaded member 2200. The agent delivery device 128 includes a device housing 140, an agent delivery control unit 146, one or more agents 162, and an electromagnetic exit port 2300 that is shown in the open position.

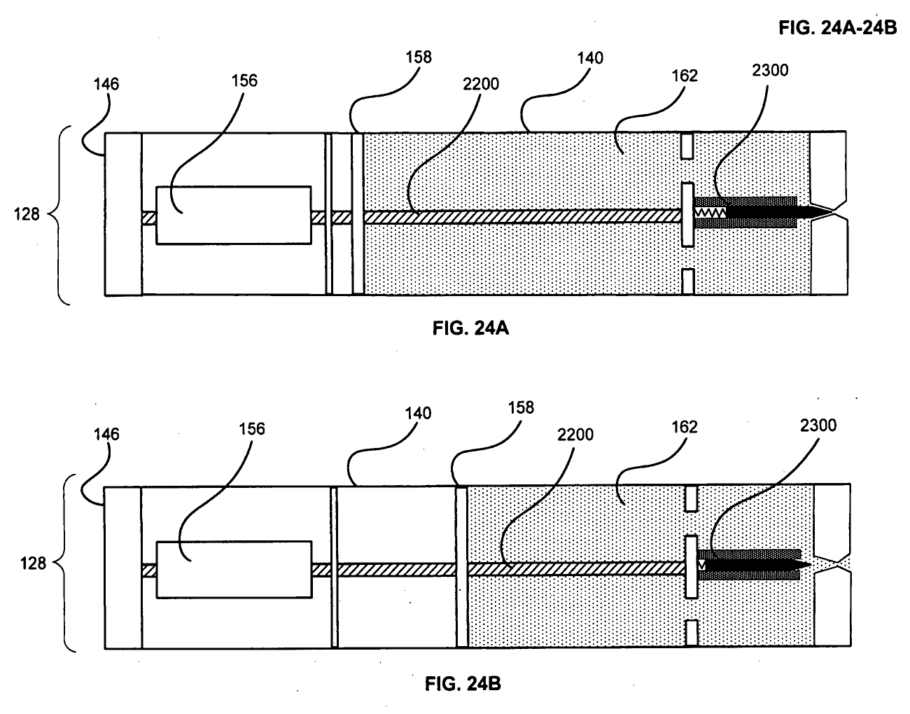

FIG. 24A illustrates an embodiment of an agent delivery device 128 that includes a squiggle motor 156 that is operably coupled to a moveable member 158 through a threaded member 2200. The agent delivery device 128 includes a device housing 140, an agent delivery control unit 146, one or more agents 162, and an electromagnetic exit port 2300 that is shown in the closed position.

FIG. 24B illustrates an embodiment of an agent delivery device 128 that includes a squiggle motor 156 that is operably coupled to a moveable member 158 through a threaded member 2200. The agent delivery device 128 includes a device housing 140, an agent delivery control unit 146, one or more agents 162, and an electromagnetic exit port 2300 that is shown in the open position.

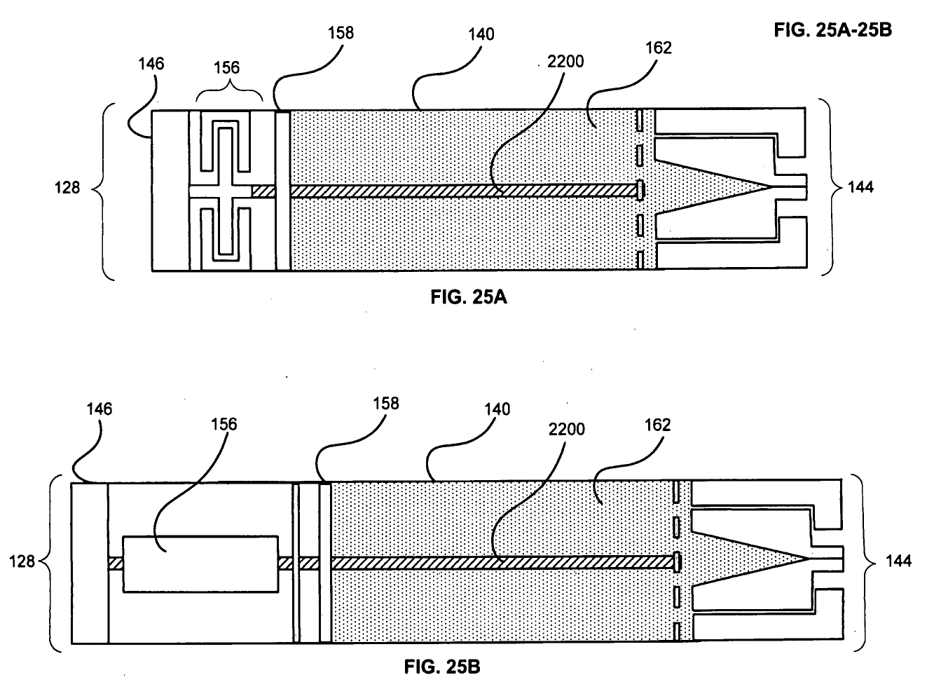

FIG. 25A illustrates an embodiment of an agent delivery device 128 that includes a stepper motor 156 that is operably coupled to a moveable member 158 through a threaded member 2200. The agent delivery device 128 includes a device housing 140, an agent delivery control unit 146, one or more agents 162, and a unidirectional exit port 144.

FIG. 25B illustrates an embodiment of an agent delivery device 128 that includes a squiggle motor 156 that is operably coupled to a moveable member 158 through a threaded member 2200. The agent delivery device 128 includes a device housing 140, an agent delivery control unit 146, one or more agents 162, and a unidirectional exit port 144.

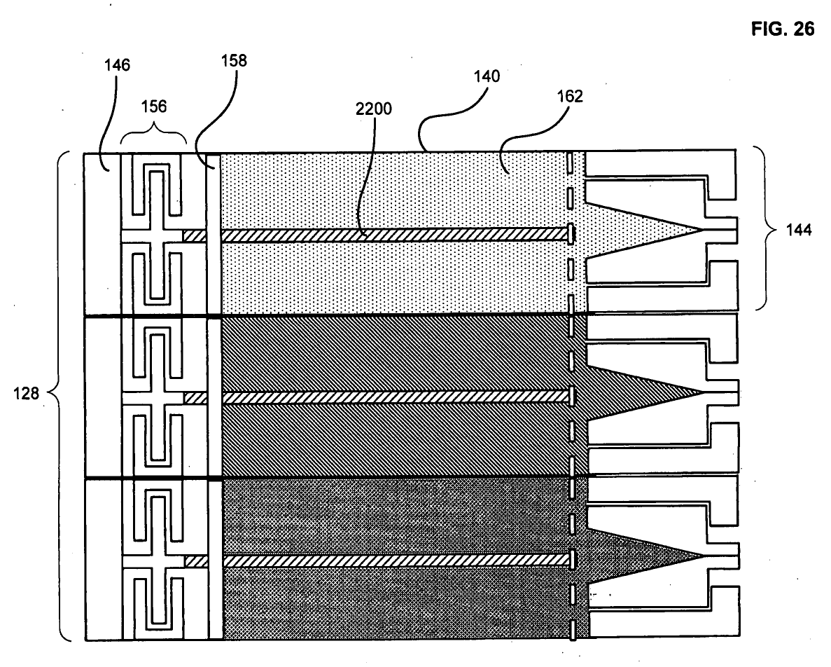

FIG. 26 illustrates an embodiment of an agent delivery device 128 that includes an assembly of agent delivery devices 128. Each of the agent delivery devices 128 includes a stepper motor 156 that is operably coupled to a moveable member 158 through a threaded member 2200. Each of the agent delivery devices 128 includes a device housing 140, an agent delivery control unit 146, one or more agents 162, and a unidirectional exit port 144.

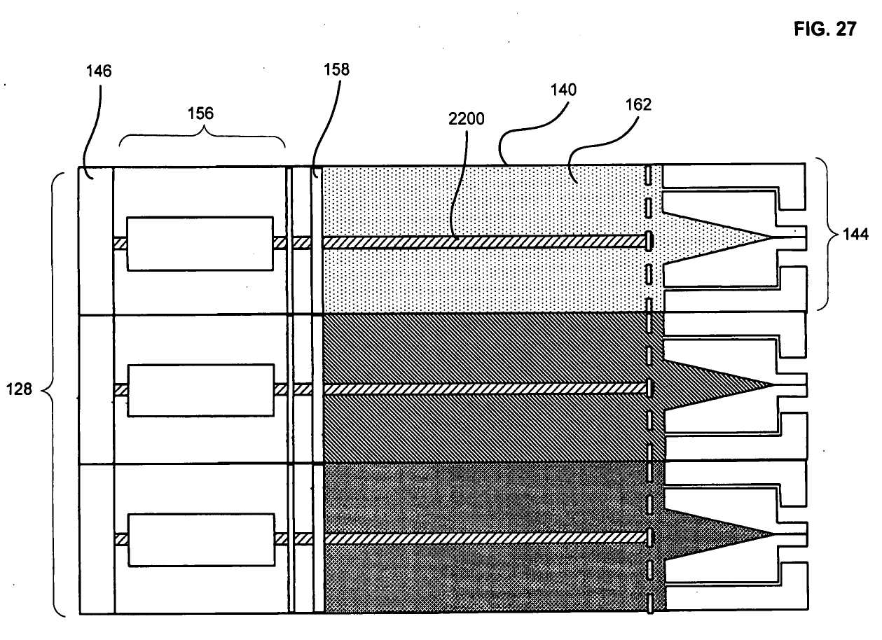

FIG. 27 illustrates an embodiment of an agent delivery device 128 that includes an assembly of agent delivery devices 128. Each of the agent delivery devices 128 includes a squiggle motor 156 that is operably coupled to a moveable member 158 through a threaded member 2200. Each of the agent delivery devices 128 includes a device housing 140, an agent delivery control unit 146, one or more agents 162, and a unidirectional exit port 144.

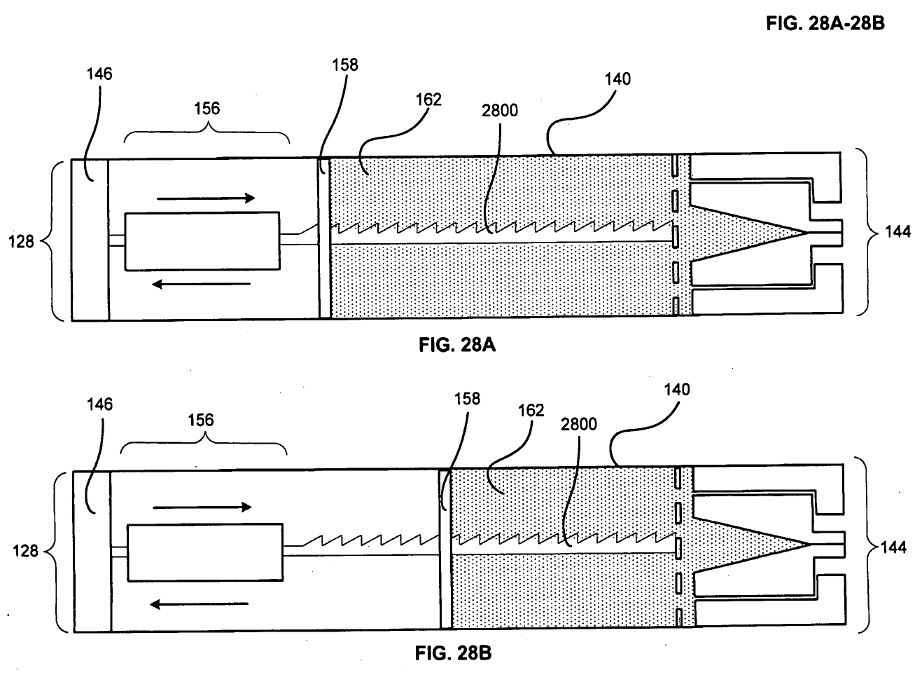

FIG. 28A illustrates an embodiment of an agent delivery device 128 that includes a piezoelectric linear motor 156 that is operably coupled to a moveable member 158 through a ratcheted member 2800. The agent delivery device 128 includes a device housing 140, an agent delivery control unit 146, one or more agents 162, and a unidirectional exit port 144.

FIG. 28B illustrates an embodiment of an agent delivery device 128 that includes a piezoelectric linear motor 156 that is operably coupled to a moveable member 158 through a ratcheted member 2800. The agent delivery device 128 includes a device housing 140, an agent delivery control unit 146, one or more agents 162, and a unidirectional exit port 144. The moveable member 158 is illustrated in an advanced position relative to the position of the moveable member 158 as illustrated in FIG. 28A.

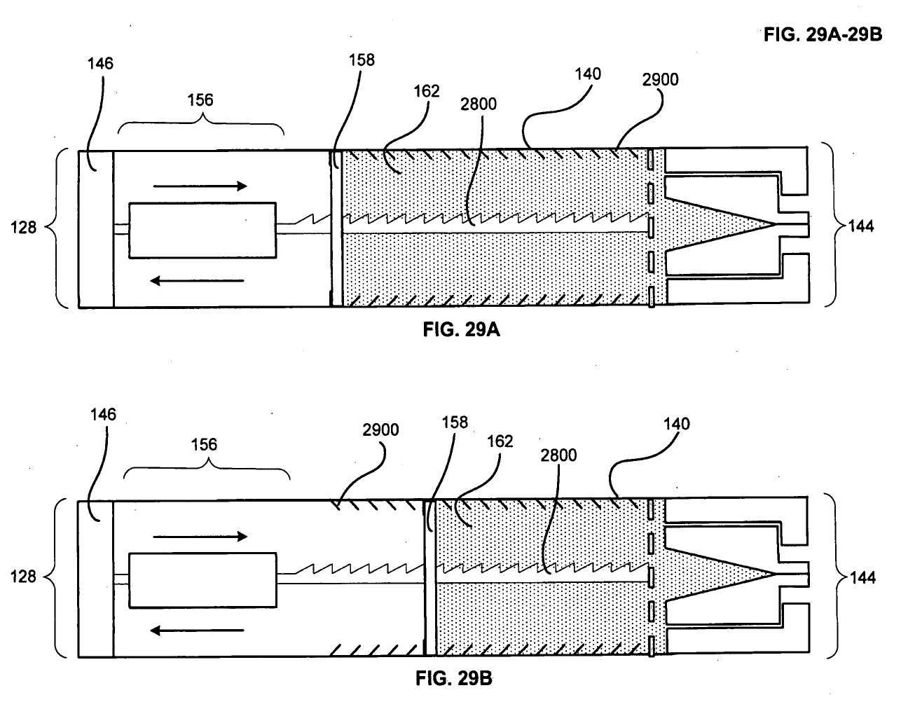

FIG. 29A illustrates an embodiment of an agent delivery device 128 that includes a piezoelectric linear motor 156 that is operably coupled to a moveable member 158 through a ratcheted member 2800. The agent delivery device 128 includes a device housing 140, an agent delivery control unit 146, one or more agents 162, a unidirectional exit port 144, and moveable member retainers 2900.

FIG. 29B illustrates an embodiment of an agent delivery device 128 that includes a piezoelectric linear motor 156 that is operably coupled to a moveable member 158 through a ratcheted member 2800. The agent delivery device 128 includes a device housing 140, an agent delivery control unit 146, one or more agents 162, a unidirectional exit port 144, and moveable member retainers 2900. The moveable member 158 is illustrated in an advanced position relative to the position of the moveable member 158 as illustrated in FIG. 29A.

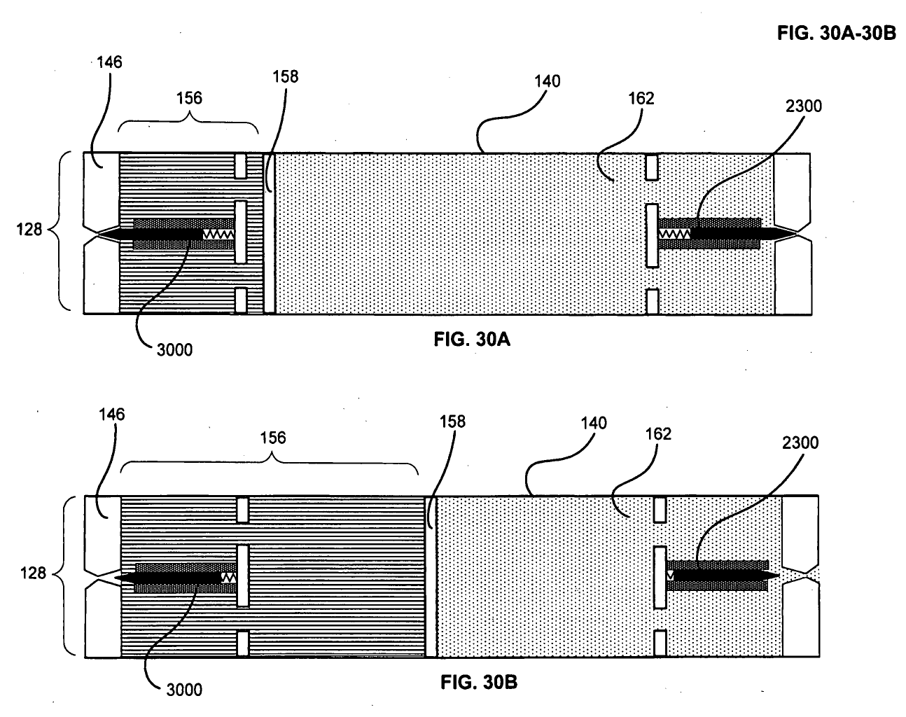

FIG. 30A illustrates an embodiment of an agent delivery device 128 that includes an osmotic motor 156 that facilitates movement of a moveable member 158 through introduction of solute into the osmotic motor 156. The agent delivery device 128 includes a device housing 140, an agent delivery control unit 146, one or more agents 162, an electromagnetic exit port 2300 that is shown in the closed position, and an electromagnetic entry port 3000 that is shown in the closed position.

FIG. 30B illustrates an embodiment of an agent delivery device 128 that includes an osmotic motor 156 that facilitates movement of a moveable member 158 through introduction of solute into the osmotic motor 156. The agent delivery device 128 includes a device housing 140, an agent delivery control unit 146, one or more agents 162, an electromagnetic exit port 2300 that is shown in the open position, and an electromagnetic entry port 3000 that is shown in the open position. The moveable member 158 is illustrated in an advanced position relative to the position of the moveable member 158 as illustrated in FIG. 30A.

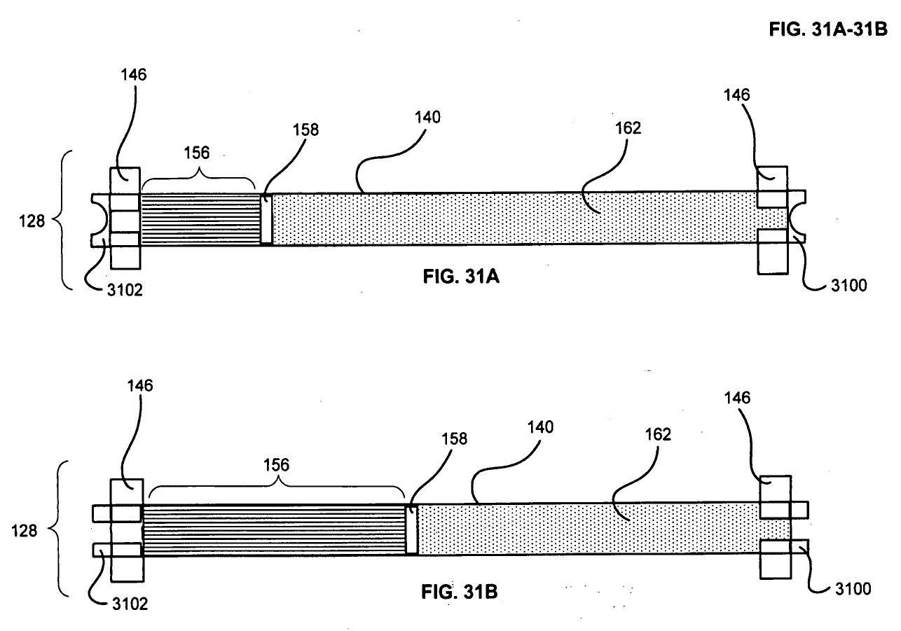

FIG. 31A illustrates an embodiment of an agent delivery device 128 that includes an osmotic motor 156 that facilitates movement of a moveable member 158 through introduction of solute into the osmotic motor 156. The agent delivery device 128 includes a device housing 140, agent delivery control units 146, one or more agents 162, an exit port 3100 made from a shape memory material that is shown in the closed position, and an entry port 3102 made from a shape memory material that is shown in the closed position.

FIG. 31B illustrates an embodiment of an agent delivery device 128 that includes an osmotic motor 156 that facilitates movement of a moveable member 158 through introduction of solute into the osmotic motor 156. The agent delivery device 128 includes a device housing 140, agent delivery control units 146, one or more agents 162, an exit port 3100 made from a shape memory material that is shown in the open position, and an entry port 3102 made from a shape memory material that is shown in the open position. The moveable member 158 is illustrated in an advanced position relative to the position of the moveable member 158 as illustrated in FIG. 31A.

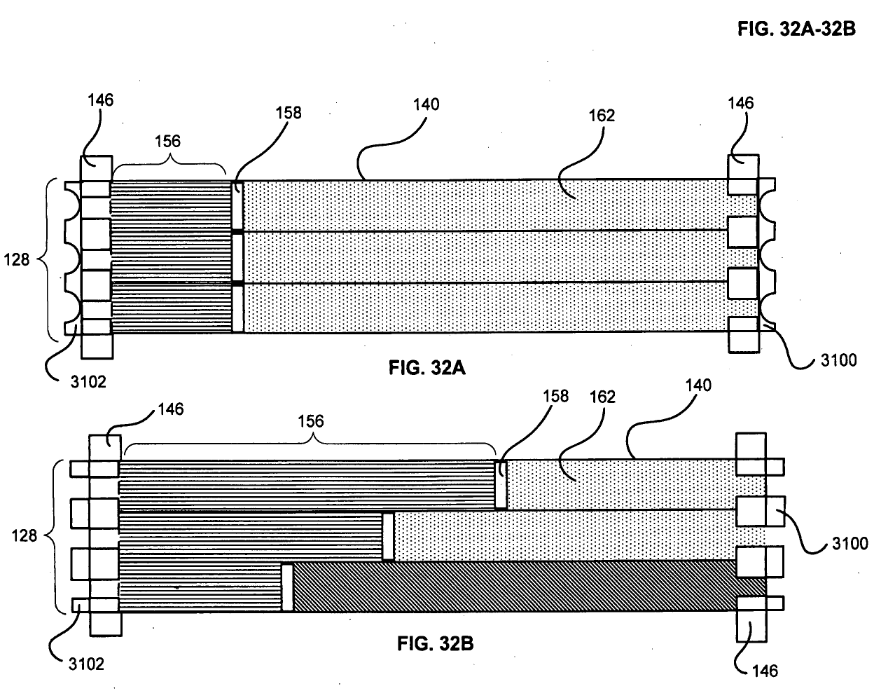

FIG. 32A illustrates an embodiment of an agent delivery device 128 that includes an assembly of individual agent delivery devices 128. Each agent delivery device 128 includes an osmotic motor 156 that facilitates movement of a moveable member 158 through introduction of solute into the osmotic motor 156. Each agent delivery device 128 includes a device housing 140, agent delivery control units 146, one or more agents 162, an exit port 3100 made from a shape memory material that is shown in the closed position, and an entry port 3102 made from a shape memory material that is shown in the closed position.

FIG. 32B illustrates an embodiment of an agent delivery device 128 that includes an assembly of individual agent delivery devices 128. Each agent delivery device 128 includes an osmotic motor 156 that facilitates movement of a moveable member 158 through introduction of solute into the osmotic motor 156. Each agent delivery device 128 includes a device housing 140, agent delivery control units 146, one or more agents 162, an exit port 3100 made from a shape memory material that is shown in the open position, and an entry port 3102 made from a shape memory material that is shown in the open position. The moveable members 158 are shown in an advanced position relative to their position as illustrated in FIG. 32A.

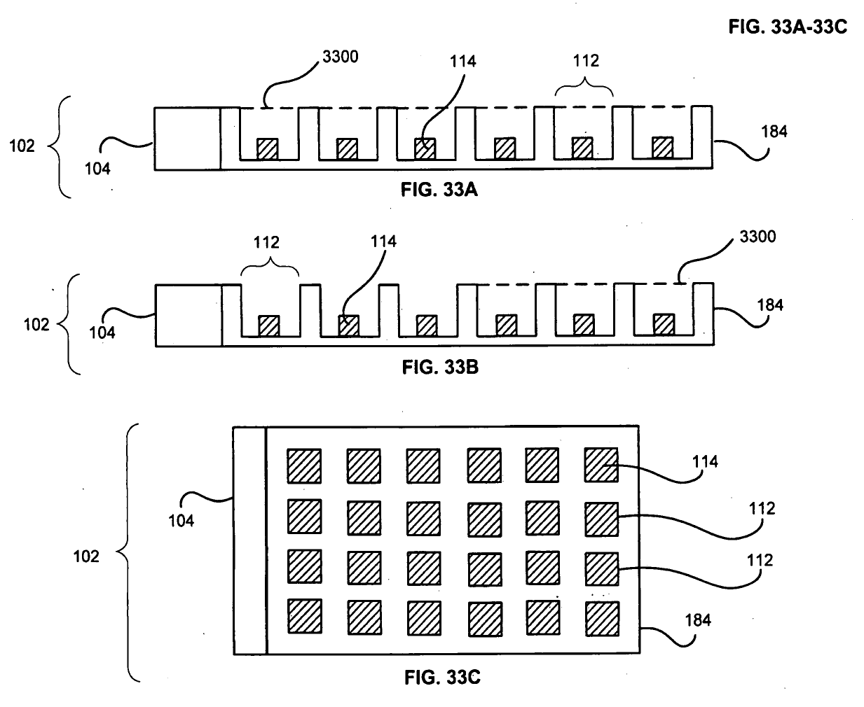

FIG. 33A illustrates a side-view of an embodiment of sensor 102 that includes a sensor control unit 104, and a sensor housing 184 that includes selectively accessible sections 112 that are covered with a sacrificial layer 3300 and which enclose sensor detectors 114. All of the selectively accessible sections 112 are shown as being sequestered from the outside environment.

FIG. 33B illustrates a side-view of an embodiment of sensor 102 that includes a sensor control unit 104, and a sensor housing 184 that includes selectively accessible sections 112 that are covered with a sacrificial layer 3300 and which enclose sensor detectors 114. The sacrificial layer 3300 is shown as having been removed from three of the selectively accessible sections 112 of the sensor 102 to expose three sensor detectors 114 to the outside environment.

FIG. 33C illustrates a top-view of an embodiment of sensor 102 that includes a sensor control unit 104, and a sensor housing 184 that includes selectively accessible sections 112 that enclose sensor detectors 114.

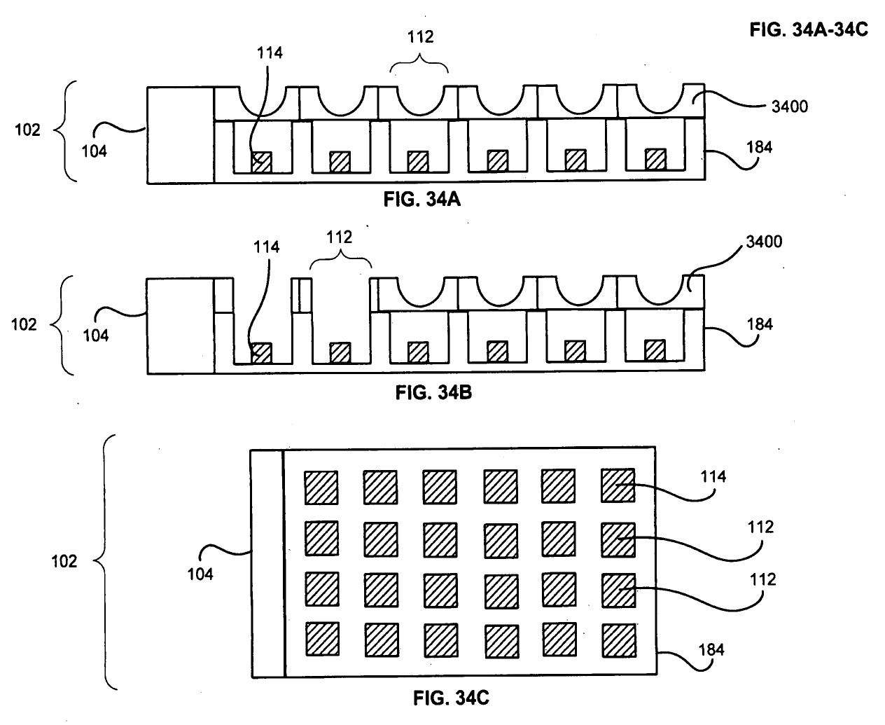

FIG. 34A illustrates a side-view of an embodiment of sensor 102 that includes a sensor control unit 104, and a sensor housing 184 that includes selectively accessible sections 112 that are covered with a shape memory material 3400 and which enclose sensor detectors 114. All of the selectively accessible sections 112 are shown as being sequestered from the outside environment.

FIG. 34B illustrates a side-view of an embodiment of sensor 102 that includes a sensor control unit 104, and a sensor housing 184 that includes selectively accessible sections 112 that are covered with a shape memory material 3400 and which enclose sensor detectors 114. The shape memory material 3400 covering two of the selectively accessible sections 112 is shown as having been reshaped to expose two sensor detectors 114 to the outside environment.

FIG. 34C illustrates a top-view of an embodiment of sensor 102 that includes a sensor control unit 104, and a sensor housing 184 that includes selectively accessible sections 112 and which enclose sensor detectors 114.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into an image processing system. Those having skill in the art will recognize that a typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems. Those skilled in the art will recognize that at least a portion of the devices and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory). A sale of a system or method may likewise occur in a territory even if components of the system or method are located and/or used outside the territory. Further, implementation of at least part of a system for performing a method in one territory does not preclude use of the system in another territory.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Those skilled in the art will appreciate that a user may be representative of a human user, a robotic user (e.g., computational entity), and/or substantially any combination thereof (e.g., a user may be assisted by one or more robotic agents) unless context dictates otherwise.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise. While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1. A sensor comprising:
one or more sensor housings that include one or more selectively accessible sections;
one or more detectors operably associated with the one or more selectively accessible sections;
one or more barriers operably associated with the one or more selectively accessible sections;
one or more sensor control units; and
one or more sensor transmitters.

2. The sensor of claim 1, wherein the one or more sensor housings that include one or more selectively accessible sections comprise:
one or more sensor housings that are configured for association with a genital region of an individual.

3. The sensor of claim 1, wherein the one or more sensor housings that include one or more selectively accessible sections comprise:
one or more sensor housings that are configured for association with a vascular region of an individual.

4-6. (canceled)

7. The sensor of claim 1, wherein the one or more sensor housings that include one or more selectively accessible sections comprise:
one or more sensor housings that are configured in association with one or more stents.

8. The sensor of claim 1, wherein the one or more detectors operably associated with the one or more selectively accessible sections comprise:
one or more osmotic detectors.

9. (canceled)

10. The sensor of claim 1, wherein the one or more detectors operably associated with the one or more selectively accessible sections comprise:
one or more cantilever detectors.

11-13. (canceled)

14. The sensor of claim 1, wherein the one or more detectors operably associated with the one or more selectively accessible sections comprise:
one or more detectors configured to detect nitric oxide.

15. The sensor of claim 1, wherein the one or more detectors operably associated with the one or more selectively accessible sections comprise:
one or more detectors configured to detect glucose

16. The sensor of claim 1, wherein the one or more detectors operably associated with the one or more selectively accessible sections comprise:
one or more detectors configured to detect one or more hormones.

17. (canceled)

18. The sensor of claim 1, wherein the one or more detectors operably associated with the one or more selectively accessible sections comprise:
one or more detectors configured to detect one or more pharmaceutical agents.

19-21. (canceled)

22. The sensor of claim 1, wherein the one or more detectors operably associated with the one or more selectively accessible sections comprise:
one or more detectors configured to detect one or more cytokines.

23. (canceled)

24. The sensor of claim 1, wherein the one or more detectors operably associated with the one or more selectively accessible sections comprise:
one or more detectors configured to detect one or more blood constituents.

25. The sensor of claim 1, wherein the one or more detectors operably associated with the one or more selectively accessible sections comprise:
one or more detectors configured to detect one or more pathogen indicators.

26. The sensor of claim 1, wherein the one or more barriers operably associated with the one or more selectively accessible sections comprise:
one or more barriers that include one or more shape memory materials.

27. The sensor of claim 1, wherein the one or more barriers operably associated with the one or more selectively accessible sections comprise:
one or more barriers that include one or more sacrificial materials.

28-29. (canceled)

30. The sensor of claim 1, wherein the one or more sensor control units comprise:
one or more sensor control units that include program instructions.

31. (canceled)

32. The sensor of claim 1, wherein the one or more sensor control units comprise:
one or more sensor control units that are configured to control one or more barriers.

33. The sensor of claim 1, wherein the one or more sensor control units comprise:
one or more sensor control units that are configured to process information acquired from the one or more detectors.

34-35. (canceled)

36. The sensor of claim 1, wherein the one or more sensor control units comprise:
one or more sensor control units that are configured to unmask one or more selectively accessible sections in response to one or more data points.

37. The sensor of claim 1, wherein the one or more sensor transmitters comprise:
one or more sensor transmitters configured to transmit one or more acoustic signals.

38. (canceled)

39. The sensor of claim 1, wherein the one or more sensor transmitters comprise:
one or more sensor transmitters configured to transmit one or more optical signals.

40-41. (canceled)

42. The sensor of claim 1, wherein the one or more sensor transmitters comprise:
one or more sensor transmitters configured to transmit one or more radio frequency signals.

43. (canceled)

44. The sensor of claim 1, wherein the one or more sensor transmitters comprise:
one or more sensor transmitters configured to transmit one or more ultrasonic signals.

45. The sensor of claim 1, wherein the one or more sensor transmitters comprise:
one or more sensor transmitters configured to transmit one or more signals according to one or more schedules.

46. The sensor of claim 1, wherein the one or more sensor transmitters comprise:
one or more sensor transmitters configured to transmit one or more signals in response to detection of one or more analytes.

47. The sensor of claim 1, wherein the one or more sensor transmitters comprise:
one or more sensor transmitters configured to transmit one or more signals in response to one or more queries.

48. The sensor of claim 1, wherein the one or more sensor transmitters comprise:
one or more sensor transmitters configured to transmit one or more encrypted signals.

49. The sensor of claim 1, further comprising:
one or more sensor receivers.

50. The sensor of claim 49, wherein the one or more sensor receivers comprise:
one or more sensor receivers configured to receive one or more acoustic signals.

51-54. (canceled)

55. The sensor of claim 49, wherein the one or more sensor receivers comprise:
one or more sensor receivers configured to receive one or more radio frequency signals.

56. (canceled)

57. The sensor of claim 49, wherein the one or more sensor receivers comprise:
one or more sensor receivers configured to receive one or more ultrasonic signals.

58. The sensor of claim 49, wherein the one or more sensor receivers comprise:
one or more sensor receivers configured to receive one or more signals according to one or more schedules.

59. The sensor of claim 49, wherein the one or more sensor receivers comprise:
one or more sensor receivers configured to receive one or more signals in response to detection of one or more analytes.

60. The sensor of claim 49, wherein the one or more sensor receivers comprise:
one or more sensor receivers configured to receive one or more signals in response to one or more queries.

61. A system comprising:
circuitry for operating one or more sensor housings that include one or more selectively accessible sections;
circuitry for operating one or more detectors operably associated with the one or more selectively accessible sections;
circuitry for operating one or more barriers operably associated with the one or more selectively accessible sections;
circuitry for operating one or more sensor control units; and
circuitry for operating one or more sensor transmitters.

62-106. (canceled)

107. The system of claim 61, further comprising:
circuitry for operating one or more sensor receivers.

108-118. (canceled)

119. A sensor system comprising:
means for operating one or more sensor housings that include one or more selectively accessible sections;
means for operating one or more detectors operably associated with the one or more selectively accessible sections;
means for operating one or more barriers operably associated with the one or more selectively accessible sections;
means for operating one or more sensor control units; and
means for operating one or more sensor transmitters.

120. The system of claim 119, further comprising:
means for operating one or more sensor receivers.

121. A system comprising:
a signal-bearing medium bearing:
one or more instructions for operating one or more sensor housings that include one or more selectively accessible sections;
one or more instructions for operating one or more detectors operably associated with the one or more selectively accessible sections;
one or more instructions for operating one or more sensor control units; and
one or more instructions for operating one or more sensor transmitters.

122. The system of claim 121, further comprising:
one or more instructions for operating one or more sensor receivers.

123-125. (canceled)
